(19) United States
(12) Patent Application Publication
(10) Pub. No.: US 2023/0031122 A1
(43) Pub. Date: Feb. 2, 2023

(54) MAGNETOELASTIC MICROCARRIERS AND MONITORING SYSTEM

(71) Applicants: Keat Ghee Ong, Eugene, OR (US); Robert E. Guldberg, Eugene, OR (US); William Skinner, Eugene, OR (US); Salil S. Karipott, Eugene, OR (US); University of Oregon, Eugene, OR (US)

(72) Inventors: Keat Ghee Ong, Eugene, OR (US); Robert E. Guldberg, Eugene, OR (US); William Skinner, Eugene, OR (US); Salil S. Karipott, Eugene, OR (US)

(21) Appl. No.: 17/789,707

(22) PCT Filed: Dec. 31, 2020
(86) PCT No.: PCT/US2020/067684

§ 371 Date: Jun. 28, 2022

Related U.S. Application Data
(60) Provisional application No. 62/956,076, filed on Dec. 31, 2019.

(51) Int. Cl. G01N 33/487; G01N 27/74; C12N 11/14; C12N 13/00; C12M 1/42; C12M 1/12

(52) U.S. Cl. CPC G01N 33/48735; G01N 27/74; C12N 11/14; C12N 13/00; C12M 35/06; C12M 25/14

(57) ABSTRACT

Certain examples of the disclosure concern an apparatus including a microcarrier configured to receive and support attachment and growth of cells, and a magnetoelastic sensor enclosed by the microcarrier. The magnetoelastic sensor is configured to vibrate in response to an activation magnetic field, and the vibration can produce a return magnetic field having detectable field characteristics associated with the attachment or growth of the cells.

### CROSS-REFERENCE TO RELATED APPLICATION

This application claims the benefit of U.S. Provisional Patent Application No. 62/956,076, filed Dec. 31, 2019, which is incorporated herein by reference.

### ACKNOWLEDGMENT OF GOVERNMENT SUPPORT

This invention was made with government support under National Science Foundation Engineering Research Center for Cell Manufacturing Technologies (CMaT), grant number 1648035. The government has certain rights in the invention.

### FIELD

The present disclosure concerns examples of magnetoelastic sensors and related systems and methods for monitoring cell culture, as well as methods of fabricating the magnetoelastic sensors.

### BACKGROUND

Cell therapies can improve the prognosis of many once untreatable diseases. Although many research projects, clinical trials, and business activities have been pursued for cell therapies, there are significant challenges to consistently and effectively assess cell number/volume and cell quality/microenvironment during the cell manufacturing process. In particular, existing technology for the tracking of cell number and monitoring of local microenvironments of the cells in a bioreactor often lead to the destruction of cells and increase the chances of contamination for a batch. Thus, there is a need for improved apparatus, systems, and methods that allow for non-intrusive and continuous monitoring of cell growth in a bioreactor.

### SUMMARY

Certain examples of the disclosure concern an apparatus including a microcarrier configured to receive and support attachment and growth of cells, and a magnetoelastic sensor enclosed by the microcarrier. The magnetoelastic sensor is configured to vibrate in response to an activation magnetic field, and the vibration can produce a return magnetic field having detectable field characteristics associated with the attachment or growth of the cells

Certain examples of the disclosure also concern an apparatus including a bioreactor chamber configured to grow cells and a microcarrier disposed inside the bioreactor chamber configured to support attachment and growth of the cells. The microcarrier can include a magnetoelastic sensor configured to vibrate with a specific resonant frequency when activated by a first magnetic field and produce a remotely detectable second magnetic field from the vibration.

Certain examples of the disclosure further concern an apparatus including a microcarrier having a magnetoelastic substrate, a first coated layer encapsulating the magnetoelastic substrate and having a biocompatible material, and a second coated layer encapsulating the first coated layer. The second coated layer can be configured to promote cells to attach to and grow on the microcarrier.

Certain examples of the disclosure concern a system for monitoring cell growth in a bioreactor. The system includes a drive coil configured to generate a first magnetic field to activate a magnetoelastic sensor inside the bioreactor, a detection coil configured to detect a second magnetic field generated by the magnetoelastic sensor after activated by the first magnetic field, and a processing unit configured to analyze the second magnetic field to calculate one or more resonant response parameters of the magnetoelastic sensor.

Certain examples of the disclosure concern a method of fabricating a cell sensing apparatus. The method includes embedding a magnetoelastic sensor inside a microcarrier configured for cells to attached thereto. The magnetoelastic sensor can be configured to vibrate with a specific resonant response when exposed to a first magnetic field and generate a second magnetic field.

Certain examples of the disclosure also concern a method for monitoring cells in a bioreactor. The method includes placing a microcarrier inside the bioreactor, wherein the microcarrier can be configured for cells to attach thereto and embed a magnetoelastic sensor. The method can further include exposing the microcarrier to a first magnetic field so that the magnetoelastic sensor vibrates with a resonant frequency and generate a second magnetic field, and detecting the second magnetic field and measuring a resonant response of the magnetoelastic sensor.

The foregoing and other objects, features, and advantages of the disclosed technologies will become more apparent from the following detailed description, which proceeds with reference to the accompanying figures.

### BRIEF DESCRIPTION OF THE DRAWINGS

FIG. 1 is a schematic depiction of a magnetoelastic sensor which can be excited by a first magnetic field and generate a second magnetic field that can be remotely detected.

FIG. 2A is a schematic depiction of an exemplary magnetoelastic sensor embedded inside a microcarrier.

FIG. 2B schematically illustrates activation of the magnetoelastic sensor to generate a mechanical loading to the microcarrier of FIG. 2A.

FIG. 3A illustrates three microcarriers, each of which is embedded with a magnetoelastic sensor of different length, according to one example.

FIG. 3B illustrates a microcarrier embedded with three magnetoelastic sensors of different lengths, according to one example.

FIG. 4A illustrates a magnetoelastic sensor with a coated layer, according to one example.

FIG. 4B illustrates a magnetoelastic sensor with multiple coated layers, according to one example.

FIG. 4C illustrates a magnetoelastic sensor with multiple coated layers, according to another example.

FIG. 5 is a plot depicting a calibration curved obtained in an experiment.

FIG. 6 schematically illustrates a 2D bioreactor and a magnetic coil system, according to one example.

FIG. 7 schematically illustrates a 3D bioreactor and a magnetic coil system, according to one example.

FIG. 8 schematically illustrates a magnetoelastic sensor monitoring system, according to one example.

FIG. 9 is a block diagram illustrating a monitoring system for a bioreactor, according to one example.

FIG. 10 schematically illustrates the concept of synthesizing a multi-frequency activation signal to drive multiple magnetoelastic sensors and extracting each sensor's frequency response from a mixed signal containing return magnetic field signals generated by all magnetoelastic sensors.

FIG. 11 is a block diagram of an example circuit for generating a multi-frequency activation signal to drive multiple magnetoelastic sensors.

FIG. 12 is a block diagram of an example circuit for extracting frequency responses of multiple magnetoelastic sensors from a mixed return magnetic field signal based on time-domain analysis.

FIG. 13 is a block diagram of an example circuit for extracting frequency responses of multiple magnetoelastic sensors from a mixed return magnetic field signal based on frequency-domain analysis.

FIG. 14 shows an example experimental result illustrating the effect of changing DC biasing field on the resonant response of a magnetoelastic sensor.

FIG. 15 shows an example experimental result depicting an impedance spectrum obtained when exposing a magnetoelastic sensor to an activation magnetic field.

FIG. 16A shows an example experimental result depicting no change in the phase of measured impedance at the resonant frequency for magnetoelastic sensors placed in a media without cells for 24 hours.

FIG. 16B shows an example experimental result depicting significant change in the phase of measured impedance at the resonant frequency for magnetoelastic sensors placed in a media with a certain seeding density at 24 hours.

FIG. 17 shows example experimental results depicting changes in the phase of measured impedance at the resonant frequency for magnetoelastic sensors placed in media having cells.

FIG. 18 shows a resonant spectrum of a magnetoelastic microcarrier embedded with three magnetoelastic sensors configured for sensing pH, oxygen, and glucose.

FIG. 19A shows distribution of pH values in a simulated 2D medium.

FIG. 19B shows simulated measurement results of pH values when placing an array of magnetoelastic microcarriers of FIG. 18 in the simulated 2D medium.

FIG. 20A shows distribution of oxygen values in a simulated 2D medium.

FIG. 20B shows simulated measurement results of oxygen values when placing an array of magnetoelastic microcarriers of FIG. 18 in the simulated 2D medium.

FIG. 21A shows distribution of glucose values in a simulated 2D medium.

FIG. 21B shows simulated measurement results of glucose values when placing an array of magnetoelastic microcarriers of FIG. 18 in the simulated 2D medium.

FIG. 22 is a block diagram of an example computing system in which described examples can be implemented.

FIG. 23 is a block diagram of an example cloud computing environment that can be used in conjunction with the technologies described herein.

### DETAILED DESCRIPTION

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the terms “coupled” and “connected” generally mean electrically, electromagnetically, and/or physically (e.g., mechanically or chemically) coupled or linked and does not exclude the presence of intermediate elements between the coupled or associated items absent specific contrary language.

Directions and other relative references (e.g., inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same. As used herein, “and/or” means “and” or “or,” as well as “and” and “or.”

As used herein, the term “approximately” and “about” means the listed value and any value that is within 20% of the listed value. For example, “about 5 μm” means any value between about 4 μm and about 6 μm, inclusive.

### Overview of Cell Growth Monitoring

Stem cell therapy products have experienced an exponential growth in the past decade. This growth is attributed to the potential of cell therapy products for treatment of numerous chronic indications. However, as more stem-cell based therapies continue to enter clinical trials, the need for robust methods and consistent processes capable of providing large quantities of stem cells in a reproducible and predictable manner, while ensuring safety and highest quality of the final cell therapy product, becomes important. Within the cell manufacturing industry, the real-time measurement of cell numbers is crucial to facilitate the optimization of critical process parameters (CPPs) such as culture conditions (pH, oxygen, glucose, etc.) and length-of-culture. Current technologies employed for monitoring cell numbers during scale-up process of anchorage-dependent stem cells like mesenchymal stromal cells (MSCs) rely on measuring the DNA content or metabolic activity of the cells, which involves either detaching the cells from the cell culture substrates (like tissue culture plates or micro carriers) or labelling with fluorescent dyes for cell counting. These additional steps of manual cell handling increase the likelihood of introducing error in determining the final cell number and they do not allow real-time monitoring of cell numbers to enable automated feedback-controlled process.

Furthermore, as cells are sensitive to their microenvironments, inhomogeneity in a bioreactor can cause differences in cells (number and quality) even when they are from the same bioreactor. In fact, today the longitudinal measurement (i.e., measuring over a period of time) of cell number and quality in a bioreactor is still labor intensive, interruptive, and/or inaccurate for many bioreactors, especially for those that do not have a homogeneous cell suspension. Although some sensors have been incorporated to bioreactors for measurement of pH, oxygen, glucose, and temperature, their main focus is for batch monitoring and not local conditions of cells. Optical techniques have also been used to quantify growth medium and cell qualities. These sensors, which are typically placed at fixed locations (not throughout the whole bioreactor), are capable for quantifying the average conditions of cells but may miss local variations within bioreactors.

Therefore, it is imperative to develop technologies that allow non-invasive, rapid, in-line and in-process monitoring of cell numbers and local microenvironmental conditions during cell culture to ensure the fabrication conditions stay within the constraints throughout the whole manufacturing process and reduce the chances of process variability.

Described herein are examples of improved sensors, apparatus, systems and related methods for monitoring cell culture. As described below, one or more microcarriers embedded with magnetoelastic sensors (also referred to as “magnetoelastic microcarriers”) can be disposed inside a bioreactor equipped with a detection system to allow continuous and non-invasive monitoring of cell growth in the bioreactor. Although specific cell types are described below as representative examples, it should be understood that the same principles described herein can also be applied to many different cell types (e.g., stem cells, T cells, etc.). Likewise, although specific magnetoelastic materials are described below as representative examples for fabricating the magnetoelastic sensors, it should be understood that other magnetoelastic materials can also be used for making the magnetoelastic sensors according to the same principles described herein. Further, although pH sensing is described below as a representative example for measuring local microenvironmental conditions of a cell culture, it should be understood that other local microenvironmental conditions (e.g., concentration and/or presence of a chemical and/or a biomarker, a virus, etc.) can also be measured based on the same principles described herein.

### Example Magnetoelastic Sensors

Described herein is an improved sensor technology that can continuously measure the number of cells, chemical concentration, and/or biomarker on individual microcarriers throughout a bioreactor without disrupting the manufacturing process. Specifically, this technology is based on wireless, micro-sized magnetoelastic sensors that can be completely encapsulated in cell microcarriers.

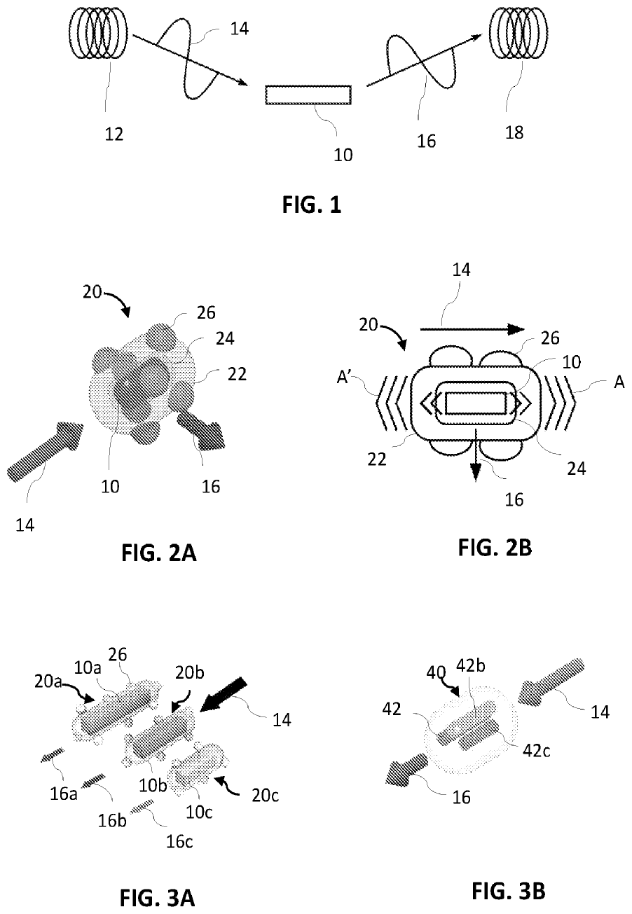

FIG. 1 schematically illustrates an operational principle of a magnetoelastic sensor 10. The magnetoelastic sensor 10 includes magnetoelastic materials, which when exposed to an alternating current (AC) magnetic field 14 (also referred to as “the first magnetic field” or “activation magnetic field” or “applied magnetic field”) generated by a drive coil 12, can produce submicron level vibrations at specific resonant frequencies and generate a secondary magnetic field 16 (also referred to as “the second magnetic field” or “return magnetic field” or “response magnetic field”) that can be remotely detected, e.g., by a detection coil 18. Thus, by activating the magnetoelastic sensor 10 with the activation magnetic field 14 through the drive coil 12 and detecting the secondary magnetic field 16 with the detection coil 18, the resonant response of the magnetoelastic sensor 10 can be remotely interrogated. Analysis of the resonant response can yield one or more resonant response parameters of the magnetoelastic sensor 10 that are indicative of various cell culture conditions within a bioreactor, as described more fully below.

The vibration amplitude and frequency of the magnetoelastic sensor 10 depend on its mechanical loadings (e.g., mass of the surrounding material). For example, the resonant frequency of the magnetoelastic sensor 10 can be determined based the following equation:

$${f}_{0} = \frac{1}{2 L} \sqrt{\frac{E}{ρ ( 1 - {σ}^{2} )}}$$

Here, the variables f₀ is the resonant frequency, L is the length of the magnetoelastic sensor 10, E and ρ are respectively the modulus of elasticity and density of the magnetoelastic sensor 10, and σ is the Poisson's ratio.

Since the resonant frequency of a magnetoelastic sensor is proportional to its length, the responses from multiple magnetoelastic sensors can be simultaneously monitored by applying an AC magnetic field that contains the frequency spectra of all resonances and then capturing the return magnetic fields. For example, the resonant frequencies of iron-nickel-molybdenum-boron (FeNiMB) magnetoelastic sensors are estimated to be 18.5 MHz, 22.2 MHz and 24.5 MHz if their lengths are 110 μm, 100 μm, and 90 μm, respectively. Thus, a frequency scan at the range of 18-25 MHz will be able to simultaneously interrogate these magnetoelastic sensors.

When the magnetoelastic sensor 10 is exposed to a compressive mechanical loading, its resonant frequency can decrease. The change of resonant frequency due to a change of mass loading on the magnetoelastic sensor 10 can be determined by the following equation.

$$Δ {f}_{0} = - {f}_{0} \frac{Δ m}{2 m}$$

where Δf₀ is the change in resonant frequency, Δm is the change in mass loading, and m is the baseline mass of the magnetoelastic sensor 10.

Similarly, the magnetoelastic sensor 10 may be functionalized by applying a coating that can shrink or swell or stretch when it is exposed to and react to a target chemical or analyte of interest, thus causing a change of loading force on the magnetoelastic sensor 10. For example, if a mass (or pressure) loading to the magnetoelastic sensor 10 increases due to absorption of a chemical or biological agent by the coating, the resonant frequency of the magnetoelastic sensor 10 can also decrease.

In addition, the magnetoelastic sensor 10 can be functionalized with chemical-sensing materials that alter their elasticity and/or viscosity in response to the analyte(s) of interest (e.g., poly(acrylic acid-co-isoocylacrylate) for pH sensing), as described more fully below. The change in the material's elasticity and/or viscosity can alter the resonance quality (e.g., the broadness of the resonance curve) of the magnetoelastic sensor 10, thus allowing independent measurement of the analyte(s).

As described herein, functionalizing a magnetoelastic sensor means equipping the magnetoelastic sensor with an additional function besides measuring a mass loading on the magnetoelastic sensor, such as detecting a biomarker around the magnetoelastic sensor, measuring a chemical concentration around the magnetoelastic sensor, measuring a temperature surrounding the magnetoelastic sensor, promoting cell adhesion to and growth on the magnetoelastic sensor, etc.

Thus, by measuring both resonant frequency and resonance quality, a microcarrier embedded with the magnetoelastic sensor 10 can be used to simultaneously measure mass loading on the microcarrier (e.g., cell adhesion) and chemical concentration around the microcarrier (e.g., with elasticity-changing chemical responsive layers).

Further, by placing multiple microcarriers in different locations of a bioreactor, remote interrogation of magnetoelastic sensors embedded in the microcarriers can wirelessly track changes in cell growth/attachment on the microcarriers and their local microenvironment over a period of time (e.g., days, weeks, or months) by measuring their respective changes in resonance profiles. Thus, the described sensor technology can monitor the whole bioreactor wirelessly, which is ideal for bioreactors that do not have a homogeneous suspension. Additionally, multiple physical/chemical quantities can be simultaneously monitored by using an array of magnetoelastic sensors that have different lengths (with different resonant frequencies) and/or are functionalized with different coating materials (also referred to as “chemical functionalization”).

### Example Microcarriers

A microcarrier is a support matrix allowing for the growth of adherent cells in a bioreactor. Microcarriers can provide anchorage or an attachment surface for the suspended cell culture, which helps in increasing the viability of the cells. Conventionally, microcarriers can come in the form of beads or other shapes and be manufactured from different materials such as gelatin, dextran, cellulose, plastic, or glass.

As described herein, microcarriers can be embedded with magnetoelastic sensors to allow the microcarriers not only function as an anchoring platform for cells to attach thereto and grow, but also allow remote, wireless, and noninvasive monitoring of cell growth in the bioreactor. Such microcarriers are also referred to as “magnetoelastic microcarriers” hereinafter.

In certain examples, a magnetoelastic microcarrier can be fabricated from scratch by coating a magnetoelastic substrate with one or more layers, as described more fully below. In other words, the magnetoelastic microcarrier can be a functionalized magnetoelastic sensor that has an outer layer that promotes cell attachment to and growth on the outer layer, just like a conventional microcarrier.

In other examples, an off-the-shelf microcarrier can be converted into a magnetoelastic microcarrier by inserting or embedding a magnetoelastic sensor therein. For example, a chamber or a recess can be created on the off-the-shelf microcarrier, and the magnetoelastic sensor can be securely placed within the chamber or recess. In some examples, the chamber or recess can be hermitically sealed. In other examples, the chamber or recess does not need to be sealed so long as the magnetoelastic sensor itself has a biocompatible coating (as described below) that insulate the magnetoelastic substrate inside the sensor from the cell culture environment.

As described herein, the magnetoelastic microcarrier has a housing configured for cells to attach thereto and grow. The housing can be an outer-most layer of the magnetoelastic sensor formed during the fabrication process as described below, or the outer shell of an off-the-shelf microcarrier that defines the chamber or recess configured to receive the magnetoelastic sensor. In some examples, the housing can be porous or have a plurality of craters on its surface so as to promote cell adhesion and anchorage onto the microcarrier.

As described herein, the magnetoelastic microcarrier is non-destructive to cells. In other words, the magnetoelastic microcarrier not only provides an anchoring surface for live cells to attach thereto, but also grow thereon. Like conventional microcarriers, the magnetoelastic microcarrier needs to promote the growth of cell culture and cannot negatively impact cell viability.

In addition, the magnetoelastic microcarrier described herein is configured to support long-term cell growth in a bioreactor, just like conventional microcarriers. Growing cells in a bioreactor can take days, weeks, or months. Thus, for longitudinal monitoring cell growth in a bioreactor, the magnetoelastic sensor embedded within the microcarrier needs to remain stable over the monitoring period. For example, the biocompatible coating of the magnetoelastic sensor needs to remain intact during the monitoring period to insulate the metallic magnetoelastic substrate from the chemical environment of the cell culture. As described herein, a longitudinal monitoring period lasts at least 1 day. In certain examples, the longitudinal monitoring period can last for 2-6 days, or 1-3 weeks, or 1-11 months, or over one year.

FIGS. 2A-2B illustrate a magnetoelastic sensor 10 embedded within a microcarrier 20. As shown, the microcarrier 20 has a housing 22, the surface of which can be configured (e.g., being porous or having a rough texture) to promote cells 26 to attach thereto and growth. The magnetoelastic sensor 10, which is embedded inside the housing 22, can have one or more coated layers or coating 24. The coating 24 can include a chemical-sensitive material, as described further below. The activation magnetic field 14 (e.g., generated by the drive coil 12) can be applied to the magnetoelastic sensor 10, causing it to expand in the direction of the activation magnetic field 14. This in turn can stretch the microcarrier 20 (as illustrated by the arrows A and A′), which leads to mechanical loading to the attached cells 26. During the activation stage, the magnetoelastic sensor 10 can generate a secondary magnetic field 16 which can be independently detected. The pattern of the secondary magnetic field 16 is dependent on the applied stress experienced by the magnetoelastic sensor 10. Thus, if the coating 24 shrinks or swells due to the changing concentration of a specific chemical, the magnetoelastic sensor 10 is exposed to different stresses and changes its return magnetic field 16, which can be remotely detected (e.g., by the detection coil 18) for detection and/or measurement of the specific chemical.

As described above, the resonant frequency of a magnetoelastic sensor depends on its length as well as the magnetoelastic material it embodies. Thus, multiple magnetoelastic sensors having different resonant frequencies (e.g., by varying magnetoelastic material and/or changing length of the sensors) can be deployed within the same bioreactor, and a frequency scan covering all resonant frequencies of the multiple magnetoelastic sensors can detect and/or measure cell loading and/or local microenvironment at each of the magnetoelastic sensors.

As an example, FIG. 3A shows three microcarriers 30a, 30b, and 30c, each of which is embedded with a respective magnetoelastic sensor 10a, 10b, and 10c. The magnetoelastic sensors 10a, 10b, and 10c have different lengths, thus having different resonant frequencies. The resonant responses from all magnetoelastic sensors 10a, 10b, and 10c can be simultaneously monitored by applying an activation magnetic field 14 that contains the frequency spectra of all resonant frequencies and then capturing the return magnetic fields 16a, 16b, and 16c. For example, if the magnetoelastic material in the sensors 10a, 10b, and 10c is iron-nickel-molybdenum-boron (FeNiMB), and the lengths of the sensors 10a, 10b, and 10c are 110 μm, 100 μm, and 90 μm, respectively, then the resonant frequencies of the magnetoelastic sensors 10a, 10b, and 10c are estimated to be 18.5 MHz, 22.2 MHz and 24.5 MHz, respective. Thus, a frequency scan at the range of 18-25 MHz will be able to simultaneously interrogate all three magnetoelastic sensors 10a, 10b, and 10c.

In some examples, the return magnetic fields 16a, 16b, and 16c can be respectively detected by multiple detection coils. In some examples, the combined magnetic fields (16a, 16b, and 16c) can be detected by one detection coil and the resonant responses from the three magnetoelastic sensors can be separated by signal processing techniques, as described further below.

As another example, FIG. 3B shows a microcarrier 40, which is embedded with three magnetoelastic sensors 42a, 42b, and 42c with different lengths (and accordingly, different resonant frequencies). Similarly, the three magnetoelastic sensors 42a, 42b, and 42c can be simultaneously monitored by applying an activation magnetic field 14 containing the frequencies of all sensors and then capturing the return magnetic fields 16 from them. For example, if the magnetoelastic sensors 42a, 42b, and 42c are made of FeNiMB alloy and their lengths are 30 μm, 34 μm, and 39 μm, respectively, then their respective resonant frequency will be about 75.9 MHz, 65.2 MHz and 56.4 MHz.

Although three magnetoelastic sensors are shown in FIGS. 3A and 3B, it should be understood that any number of magnetoelastic sensors can be embedded in any number of microcarriers. For example, tens, hundreds or thousands of microcarriers can be placed in a bioreactor, and each microcarrier can be embedded with one, two, three, or more magnetoelastic sensors. The magnetoelastic sensors embedded in different microcarriers can be the same or different, depending on applications.

### Example Bioreactor

As described herein, a bioreactor refers to a device or system designed to grow cells or tissues in the context of cell culture. The bioreactor can vary in scale. In one example, the bioreactor can be a small petri dish, ajar or bottle, or an Erlenmeyer flask typically found in a laboratory. In another example, the bioreactor can be a large volume container that can support hundreds or even thousands of liters of cell culture. In certain examples, a bioreactor can be two-dimensional (2D), e.g., when the bioreactor is configured to support 2D cell culture systems (e.g., the cells mainly grow in a 2D space, such as a plate). In other examples, a bioreactor can be three-dimensional (3D), e.g., when the bioreactor is configured to support 3D cell culture systems (e.g., the cells grow in a 3D volume defined by the bioreactor).

As described herein, one or more microcarriers embedded with magnetoelastic sensors can be placed inside a bioreactor. The number of such magnetoelastic microcarriers can vary depending on the size of the bioreactor and/or desired spatial resolution of the monitoring system. For example, the number of magnetoelastic microcarriers in a bioreactor can range from tens to hundreds, thousands, or more. The magnetoelastic microcarriers can be uniformly or non-uniformly distributed within the bioreactor. Effectively, each magnetoelastic microcarrier serves as a growth platform that not only can support the cells to grow, but is also able to assess the adhered cells (in terms of the number and quality of the cells) longitudinally and differentiate them from the non-adherent cells. As described more fully below, the bioreactor can be equipped with a monitoring system which can activate the magnetoelastic sensors inside the bioreactor and analyze the resonance response from the magnetoelastic sensors so as to detect and/or measure conditions (e.g., cell volume and/or local microenvironment) of the cell culture, and longitudinally track the cell growth inside the bioreactor.

### Example Method of Sensor Fabrication

As described herein, the magnetoelastic sensor can be constructed using a magnetoelastic substrate comprising a magnetoelastic material, such as amorphous ferromagnetic alloys (e.g., FeNiMB) or iron-based alloy (e.g., iron-gallium or FeGa). Other magnetoelastic materials can be similarly used as the substrate for the magnetoelastic sensors described herein.

In some examples, the magnetoelastic material may be commercially available with a predefined shape (e.g., rod- or ribbon-shape). For example, Metglas® 2826MB manufactured by Metglas, Inc. is a FeNiMB alloy which is commercially available as a 29 μm-thick long ribbon of 12.7 mm wide. Magnetoelastic substrates can be created by cutting such ribbon into segments having a predefined dimension. For example, the magnetoelastic substrates can be created by mechanically shearing (or laser cutting) the ribbon widthwise at every 5 mm spacing, forming a rectangular strip of 12.7 mm×5 mm. In another example, the ribbon can be cut lengthwise and then widthwise to create a rectangular strip of other desired dimensions. The cut magnetoelastic substrates can be cleaned with a cleaning solution (e.g., 2-propanol), followed by air-drying and annealing at a predefine temperature for a predefined duration (e.g., at 125° C. for 2 hours) to improve the magnetoelastic and magnetic properties of the substrates (e.g., reducing the anisotropy and/or increasing the detection sensitivity).

In some examples, magnetoelastic substrates can be created via electrodeposition and photolithography. For example, FeGa alloys with a desired atomic percentage (e.g., ranging from 70-30 to 90-10 (Fe—Ga)) can be electroplated on a polymer substrate (e.g., gold-coated polyethylene substrate) to form a layer with desired thickness (e.g., 20 μm). Then, a photoresist material can be deposited to the layer of magnetoelastic material and masking the layer of magnetoelastic material with one or more masks having a predefined shape and/or size. For example, the masks can be an array of rectangles with predefined dimensions (e.g., 20 μm wide, 90 μm-110 μm long). Next, an ultraviolet (UV) light can be applied to harden the unmasked area and the uncured photoresist can be lifted. Then, plasma etching can be used to remove the uncovered area. The remaining photoresist and the polymer substrate can be removed with organic solvents, resulting in square-rod shape magnetoelastic substrates with a predefined aspect ratio.

As described herein, the aspect ratio is calculated as a ratio of an axial length of the magnetoelastic substrate to a maximum width in a cross-section that is perpendicular to the axial length of the magnetoelastic substrate. Generally, magnetoelastic sensors are more sensitive to mass changes closer to the transverse edges of the surface, and the effect can be more profound for smaller sensors. Thus, magnetoelastic sensors are desirable to have a small dimension to improve their sensing sensitivity. As the length of the magnetoelastic sensor determines its resonant frequency, a large aspect ratio given a fixed sensor length corresponds to a smaller cross-sectional width. On the other hand, considering the manufacturing complexity and/or structural integrity, the width of the magnetoelastic substrate cannot be too small. For the magnetoelastic sensors described herein, the aspect ratio of their magnetoelastic substrates is generally greater than 1 and can range from about 1.5 and about 20. For example, the aspect ratio of the magnetoelastic substrate can be between about 1.5 and about 10, or between about 2 and about 8, or between about 2 and about 6, or between about 2 and about 4.

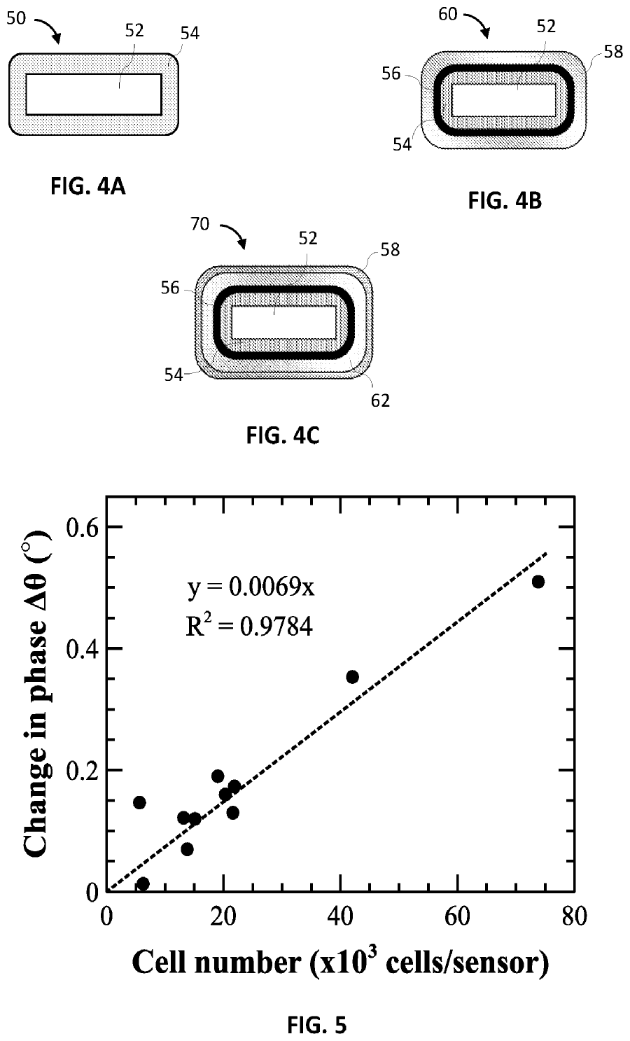

The magnetoelastic substrate described above can then be coated one or more layers to form the magnetoelastic sensor and/or magnetoelastic microcarrier, as illustrated in FIGS. 4A-4C.

FIG. 4A shows a magnetoelastic sensor 50 having a magnetoelastic substrate 52 coated with one protective layer 54. As described herein, the protective layer 54 can include a material that is biocompatible and can resist corrosion under in vitro and/or in vivo environment for the desired longitudinal monitoring period (e.g., days, weeks, months, etc.). In one specific example, the protective layer 54 can include parylene. For example, the magnetoelastic substrate 52 can be coated with a Parylene-C conformal layer with a predefined thickness (e.g., 10 μm). It should be understood that other biocompatible and corrosion-resist materials can also be used for forming the protective layer 54.

In certain examples, the magnetoelastic sensor 50 can be transformed into a magnetoelastic microcarrier by functionalizing the protective layer 54 so that it can serve as an anchoring surface for cell attachment and promote cell growth. For example, the protective layer 54 can be treated with oxygen plasma in a reactive ion etching system for a predetermined duration (e.g., 30 seconds) and at specific power level (e.g., 100 W) to improve cell adhesion by increasing the surface roughness on the protective layer 54. In certain examples, the magnetoelastic sensor 50 can be sterilized, for example, with ethanol wash followed by UV treatment for a predetermined duration (e.g., 30 minutes) on each side prior to using them in cell culture measurements.

FIG. 4B shows a magnetoelastic sensor 60 which has a magnetoelastic substrate 52 encapsulated in multiple layers. For example, the magnetoelastic substrate 52 can be encapsulated within a first protective layer 54 (e.g., parylene) to prevent corrosion and leaching, similar to the configuration shown in FIG. 4A. In addition, the magnetoelastic sensor 60 can be further coated with a second layer 56 configured to facilitate attachment of cells to the sensor without destructing the cells and promote growth of the cells. For example, the second layer 56 can include polydopamine. Polydopamine coating mimics the adhesive chemistry found in mussel adhesive proteins and can be further used to covalent link with molecules or polymers containing nucleophilic groups (e.g., —NH2). Thus, the polydopamine coated second layer 56 can act as an anchor for surface functionalization and transform the magnetoelastic sensor 60 into a magnetoelastic microcarrier.

Optionally, to promote cellular adhesion to the magnetoelastic sensor 60, an outer layer 58 can be further formed outside and bind to the second layer 56. For example, the outer layer 58 can comprise denatured collagen, which can be covalently linked to the polydopamine coating in a mildly basic solution (e.g., pH 8.5, 10 mM Tris buffer). The end result of the above fabrication process is a magnetoelastic sensor 60 that has an outer surface (or housing) that can promote cell adhesion with performance similar to (or better than) conventional microcarriers.

FIG. 4C shows another magnetoelastic sensor 70 which has similar structure as the magnetoelastic sensor 60, except for another functional layer 62 encapsulating the second layer 56. Specifically, in the depicted example, the functional layer 62 is sandwiched between the second layer 56 and the outer layer 58. The functional layer 62 can comprise a chemical configured to react to an analyte surrounding the magnetoelastic sensor 70 such that reaction of the chemical to the analyte can change elasticity and/or viscosity of the magnetoelastic sensor 70, thereby changing its resonant response. Thus, the magnetoelastic sensor 70 can be transformed into a magnetoelastic microcarrier that can detect not only cell loading, but also local microenvironment surrounding at the magnetoelastic sensor 70.

One example method of adding the functional layer 62 for pH sensing is described herein, although it should be understood that a functional layer configured for sensing other chemicals or biomarkers can be similarly constructed based on the principles described herein.

In one specific example, dopamine-functionalized atom transfer radical polymerization (ATRP) initiator can be synthesized and chemically tethered to the surface of polydopamine-coated magnetoelastic sensor (e.g., the second layer 56) through autoxidation and crosslinking of catechol found in dopamine. The initiator-coated surface can be used for surface-initiated living polymerization for grafting a block copolymer consisted of a pH-responsive inner block and an outer block for further functionalization with denatured collagen (e.g., in outer layer 58) to promote cell adhesion. For the pH-responsive block, the amount of anionic acrylic acid (10-100 mol %) can be diluted with neutral hydroxyethyl acrylate (0-90 mol %) to control the charge density and the extent elasticity change associated with pH. The pH-responsive block can also be copolymerized with 0-2 mol % of a bifunctional crosslinker (N, N′-methylenebisacrylamide) to create a covalent crosslinked network to further control its elasticity. The outer block can be polymerized using N-hydroxysuccinimide (NHS) functionalized acrylic acid, which consists of an activated carboxylate group for forming an amide bond with primary amine found on proteins. Then, denatured collagen (e.g., in the outer layer 58) can be covalently linked (e.g., to the polydopamine in the second layer 56) to create a surface that promotes cellular adhesion. In one example, the polymer volume can be determined using environmental scanning electron microscope (SEM) to estimate the changes in the Young's modulus (for calculating the magnetoelastic sensor's resonant frequency) associated with pH change.

### Example Method of Functionalizing Magnetoelastic Sensors

As described above, a magnetoelastic sensor can be functionalized with one or more layers of coating to transform the magnetoelastic sensor into a magnetoelastic microcarrier, and/or providing the magnetoelastic sensor with additional sensing capabilities besides measurement of cell loading.

The following describes the general principles for several examples of functionalizing a magnetoelastic sensor for measuring local microenvironment near the magnetoelastic sensor.

In one example, as noted above, the magnetoelastic sensor can be functionalized to detect changes in pH. Specifically, the magnetoelastic sensor can be functionalized with a pH sensitive material, such as hydrophobically modified copolymer of acrylic acid and iso-octylacrylate, a material that changes mass relative to pH. Because the magnetoelastic sensor is sensitive to changes in mass loading, such coating allows the magnetoelastic sensor to monitor pH through the same mechanism as measuring cell number, as described further below.

In another example, the magnetoelastic sensor can be functionalized to measure local glucose levels. For example, the magnetoelastic sensor can be coated with a pH-sensitive polymer (as described above) which is capable of swelling and modulating its mass in accordance with the pH of its environment. This change in mass can affect the resonant frequency of the sensor, allowing the sensor to detect changes relative to pH. This pH sensitive polymer can be further coated with a glucose sensitive material, such as glucose oxidase (GOx). The GOx is an enzyme that can convert glucose into gluconic acid, which causes the pH-sensitive polymer to shrink and lose mass, resulting in a change in resonant frequency that can be used to determine local glucose levels.

In yet another example, the magnetoelastic sensor can be functionalized to measure local CO₂ levels. For example, utilizing a similar co-polymer of acrylic acid and iso-octylacrylate (as for pH sensing), but with a different ratio, the coating on the magnetoelastic sensor can be optimized to measure and monitor levels of CO₂. As with other functionalization techniques described herein, this polymeric coating can change mass in response to the amount of CO₂ dissolved in the aqueous environment, which in turn can modulate the resonant frequency of the magnetoelastic sensor which can be detected remotely.

In yet an additional example, the magnetoelastic sensor can be functionalized to detect bacteria, such as E. coli. For example, to detect E. coli., the magnetoelastic sensor can be functionalized with a coating of immobilized E. coli. antibody as well as alkaline phosphatase. When the immobilized antibody binds to the E. coli. bacteria, the mass of the magnetoelastic sensor can changes. However, the amount of mass change may not be enough to produce desirable response activity. To compensate for this, the alkaline phosphatase enzyme can biocatalytically precipitate 5-bromo-4-chloro-3-indolyl phosphate in response the binding between the antibody and bacteria. The precipitation of this insoluble material on the surface of the magnetoelastic sensor can produce a sufficiently large mass-change that can be detected by the magnetoelastic sensor to determine the amount of E. coli present in the local microenvironment.

Other examples of analyte that can be detected by magnetoelastic sensors with specific functional coating include, but not limited to oxygen, ammonia, staphylococcal enterotoxin B, ricin, endotoxin, organophosphorus pesticide, etc.

In yet an alternative example, two magnetoelastic sensors without chemical-sensitive coating (i.e., no chemical functionalization) can be used to measure and monitor local temperature. Changes in temperature can naturally modulate the elasticity of the magnetoelastic sensor. For example, the magnetoelastic material generally becomes more elastic at warmer temperatures and less elastic at colder temperature. By employing two magnetoelastic sensor (without chemical functionalization) having different aspect ratios, two different temperature-response curves can be measured. The difference in the temperature-response curves can be used to accurately determine local temperature in a remote manner.

### Example Method of Sensor Characterization

In certain examples, the shape, size, and surface structure of fabricated magnetoelastic sensors can be characterized with SEM to ensure correct dimension and no observable defects that may impact their mechanical and magnetic properties. In some examples, magnetic characteristic of the magnetoelastic sensors can be characterized with a vibrating sample magnetometer (VSM). For example, magnetoelastic sensors, suspended in water, can be uniformly dispersed on the surface of a glass slide and allowed to dry to form a thin layer. The glass slide can be attached to the VSM sample holder. The magnetization profile (e.g., the magnetic hysteresis curve) of the magnetoelastic sensors can be characterized. The magnetoelastic property of the magnetoelastic sensors can be indirectly characterized with a detection system (similar to the monitoring system described further below), which exposes the magnetoelastic sensors with an activation magnetic field and monitor the return magnetic field signals.

### Example Method of Sensor Calibration

The fabricated magnetoelastic sensors can be calibrated for cell growth monitoring. As described above, the resonant response of a magnetoelastic sensor when exposed to an activation magnetic field depends on both the mass loading and elasticity and/or viscosity of the magnetoelastic sensor.

Thus, as the number of cells attach to the magnetoelastic sensor (or magnetoelastic microcarrier) changes, the mass loading on the magnetoelastic sensor varies, causing corresponding changes in the sensor's resonant response. Accordingly, by quantifying the number of cells (e.g., independent variable) attached to the sensor at different stages of a cell culture and measuring the sensor's resonant response (e.g., dependent variable) at respective stages, a cell volume calibration curve for the magnetoelastic sensor can be created. To control for the confounding effect caused by elasticity and/or viscosity changes on the sensor's resonant response, a constant microenvironment around the magnetoelastic sensor can be maintained during such calibration process. Based on the cell volume calibration curve, real-time longitudinal tracking of cell volume (e.g., cell count) attached to the magnetoelastic sensor (or magnetoelastic microcarrier) can be accomplished.

Similarly, when a chemical concentration adjacent the magnetoelastic sensor changes, it can affect the mass loading and/or elasticity/viscosity of the magnetoelastic sensor, causing corresponding changes in the sensor's magnetoelastic response. Thus, by varying an analyte's concentration (e.g., independent variable) around the magnetoelastic sensor while simultaneously measuring the magnetoelastic sensor's resonant response (e.g., dependent variable), a calibration curve for measuring the analyte's concentration can be established. To remove the confounding effect caused by different cell loadings, the number of cells attached to the magnetoelastic sensor can be controlled during calibration. Based on the analyte's concentration calibration curve, real-time longitudinal monitoring of the analyte's concentration surrounding the magnetoelastic sensor can be accomplished.

As an example, FIG. 5 shows a cell volume calibration curve obtained for a magnetoelastic sensor obtained during an experimental study. In this experiment, the phase of impedance at the resonant frequency, θ, was used to track the number of cells attached to the magnetoelastic sensor. As described further below, the phase of impedance at the resonant frequency (θ) can be one of a plurality of resonant response parameters that can be detected and calculated by the monitoring system and used for monitoring cell growth in a bioreactor. In the experiment, L929 fibroblast cells were seeded at varying densities ranging from 5×10³ cells/cm' to 4×10⁴ cells/cm'. Magnetoelastic sensor measurements were taken at two different time points, before (0 hr.) and after cell attachment (24 hrs.) to the sensor surface. Varying densities of the L929 fibroblast cells seeded on the magnetoelastic sensor were used to produce different cell numbers. To determine the actual cell numbers on the sensor's surface, the cells on the sensor's surface were immediately fixed after taking sensor reading at a predetermined duration of cell culture (e.g., 24 hours). The nuclei of L929 fibroblasts were then stained with Hoechst dye (DNA binding stain) and imaged with confocal microscopy, where nuclei of individual cells were annotated and quantified. The quantification of actual cell numbers, when compared to the sensor measurements (e.g., θ), allows generation of a calibration curve for L929 fibroblast cells. FIG. 5 shows a linear relationship between the sensor response (e.g., change in phase, Δθ) and the number of cells attached to the sensor surface, with R²=0.97. Assuming average mass of the fibroblast cells is 1 ng/cell, the magnetoelastic sensor used in this experiment showed a linear mass sensitivity of ˜7°/mg.

It should be understood that the calibration curve shown in FIG. 95 is merely exemplary. In some examples, the calibration curve for a magnetoelastic sensor may be based on other having a nonlinear shape. In some examples, the dependent variable for the calibration curve can be change of resonant frequency or change of impedance at the resonant frequency (instead of change of phase at the resonant frequency). In some examples, the independent variable for the calibration curve can be concentration of an analyte (instead of number of cells attached to the sensor).

### Example Magnetic Coils

As noted above, magnetoelastic sensors can vibrate in response to an activation magnetic field, and such vibration can produce a return magnetic field having detectable field characteristics associated with the attachment or growth of the cells. The activation magnetic field can be generated by a drive coil and the return magnetic field can be detected by a detection coil. In certain examples, the drive coil and the detection coil can be the same, wherein switches and/or buffers can be used isolate a drive circuit used to provide an excitation current (also referred to as “drive current”) used to generate the activation magnetic field and a detection circuit (or signal processing unit) used to detect and process the return magnetic field signal.

In some examples, the activation magnetic field can include both a direct-current (DC) magnetic field component (also referred to as the “DC biasing field”) and an AC magnetic field component. Correspondingly, the drive coil can include a direct-current (DC) coil and an AC coil. The AC coil can be connected to an AC current source to generate the AC magnetic field component, and the DC coil can be connected to a DC current source to generate the biasing magnetic field. The AC magnetic field component can activate the magnetoelastic sensors, and the DC biasing field can be configured to maximize the magnetoelastic sensor's resonance signal. Specifically, the DC biasing field can increase the magnetoelastic effect of the magnetoelastic sensors, leading to a larger resonance. Thus, by varying the magnitude of the DC biasing field, the largest change in sensor response can be predetermined and selected for following sensor measurement.

As noted above, a bioreactor can include a plurality of magnetoelastic sensors (or magnetoelastic microcarriers) that are distributed in a 2D or 3D space. As described herein, each magnetoelastic sensor can be selectively activated by the activation magnetic field generated by the drive coil and the return magnetic field generated by each magnetoelastic sensor can be selectively detected by the detection coil.

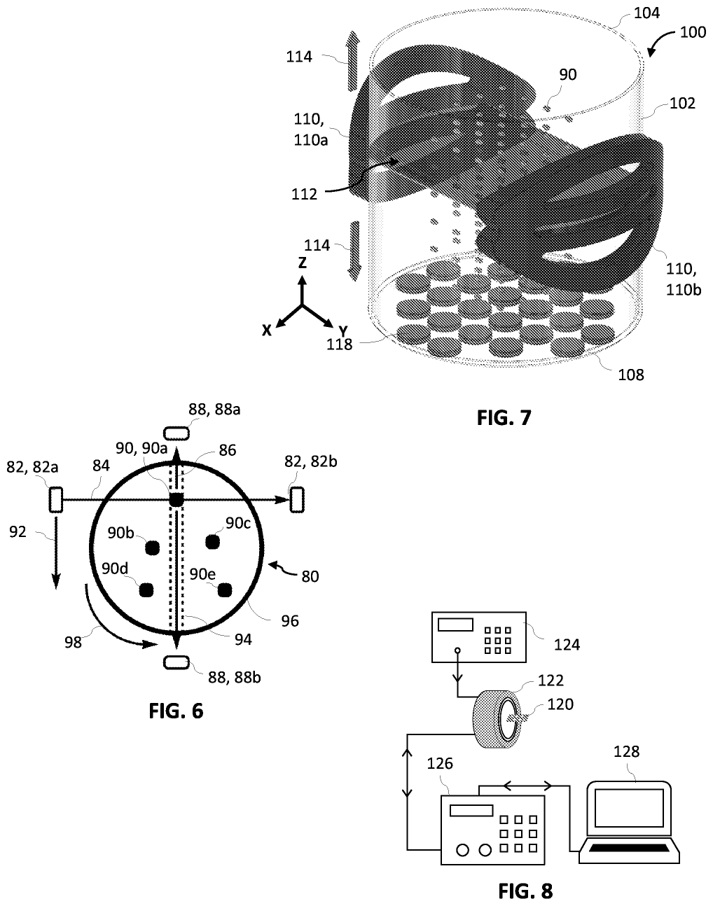

As an example, FIG. 6 schematically depicts a 2D bioreactor 80 having a chamber 96 which encloses a plurality of magnetoelastic sensors 90a, 90b, 90c, 90d, 90c (collectively, 90) that are spaced apart from each other. The magnetic coil system includes a drive coil 82 configured to generate an activation magnetic field 84 to activate the magnetoelastic sensors 90 and a detection coil 88 configured to detect the return magnetic field 86 generated by the magnetoelastic sensors 90. In the depicted example, the magnetoelastic sensors 90 are oriented so that the activation magnetic field 84 is perpendicular to the return magnetic field 86. In other examples, the activation magnetic field 84 may not be perpendicular to the return magnetic field 86.

As shown, the drive coil 82 includes a pair of drive coil conductors 82a, 82b disposed on opposite sides of the bioreactor 80, and the direction of the activation magnetic field 84 points from the drive coil conductor 82a to the drive coil conductor 82b. The detection coil 88 also includes a pair of detection coil conductors 88a, 88b located diametrically opposite to each other outside the chamber 96. To maximize the detection sensitivity, the detection coil 88 can be oriented so that the return magnetic field 86 extends through an axial axis of the detection coil 88.

To selectively activate and detect the magnetoelastic sensors 90, the drive coil 82 can move from one side of the chamber 96 to the other side in a direction 92 that is perpendicular to the activation magnetic field 84 generated by the drive coil 82. As a result, at a given time, only magnetoelastic sensors (e.g., 90a) exposed by the activation magnetic field 84 will be activated, and the detection coil 88 will only pick up the return magnetic field signal 86 from magnetoelastic sensors (e.g., 90a) located along a center region (marked by dashed lines) passing through (and normal to) the detection coil 88. The width of the center region 94 is generally related to and defined by the diameter of the detection coil 88.

In certain examples, a scan operation, e.g., by moving the drive coil 82 in the direction 92 from one side to the other side of the chamber 96, can result in selectively measurement of magnetoelastic sensors 90 located at the center region 94 of the bioreactor 80. The chamber 96 can then be slightly rotated, e.g., in counter-clockwise direction 98 (or alternatively in clockwise direction) and scanned again until the whole bioreactor is scanned (e.g., after a 180-degree rotation). Thus, the magnetoelastic sensors 90 inside the chamber 96 can be sequentially exposed to the activation magnetic field 84 by actuating relative linear movement between the drive coil 82 and the bioreactor 80 (e.g., in direction 92), and return magnetic field 86 generated by the magnetoelastic sensors 90 can be sequentially detected by the detection coil 88 by actuating relative rotation between the detection coil 88 and the bioreactor 80 (e.g., in direction 98). As such, the combination of linear movement of the drive coil 82 and rotational movement of the chamber 96 can cause each magnetoelastic sensor 90 to be located, at least for a period of time, at an intersection between the activation magnetic field 84 and the center region 94 defined by the detection coil 88, thus being selectively activated and detected.

In alternative examples, the scan operation of the bioreactor 80 can be conducted in different ways. For example, instead of moving the drive coil 82, the drive coil 82 can remain stationary while the chamber 96 can move linearly relative to the drive coil 82 in the direction 92. In another example, instead of rotating the chamber 96, the chamber 96 can be nonrotational while the detection coil 88 can be configured to rotate circumferentially relative to the chamber 96. In other words, so long as the drive coil 82 and the chamber 96 can move linearly relative to each other and the detection coil 88 and the chamber 96 can rotate relative to each other, a 2D scan of all magnetoelastic sensors 90 in the bioreactor 80 can be accomplished.

In other examples, the scan operation of the bioreactor 80 can be accomplished without relative movement between the bioreactor 80 and the drive coil 82 and/or the detection coil 88. For example, instead of relative linear movement between the drive coil 82 and the chamber 96, the drive coil 82 can comprise a plurality of pairs of drive coil conductors 82a, 82b that are linearly distributed along the direction 92 so that these pairs of drive coil conductors can be selectively activated to generate the activation magnetic field 84 at different part of the bioreactor 80. Likewise, instead of relative rotation movement between the detection coil 88 and the chamber 96, the detection coil 88 can comprise a plurality of pairs of detection coil conductors 88a, 88b that are distributed around the circumference of the chamber 96 so that these pairs of detection coil conductors can selectively pickup return magnetic field signals generated by different magnetoelastic sensors 90 inside the bioreactor 80.

In the depicted example, the activation magnetic field 84 is perpendicular to the return magnetic field 86, and the drive coil conductors 82a, 82b are symmetric about an imaginary line connecting the detection coil conductors 88a, 88b. In other examples, the activation magnetic field 84 may not be perpendicular to the return magnetic field 86, and the drive coil conductors 82a, 82b may be asymmetric about the line connecting the detection coil conductors 88a, 88b.

In the depicted example, the surface of the bioreactor 80 has a circular shape. In other examples, the surface of the bioreactor 80 can have a non-circular shape, such as a rectangle, a square, an ellipse, a polygon, etc. In certain examples, the detection coil 88 may include only one detection coil conductor, e.g., 88a or 88b.

As another example, FIG. 7 schematically illustrates a 3D bioreactor 100 which encloses a plurality of magnetoelastic sensors 90 (or magnetoelastic microcarriers) distributed within the 3D space of the bioreactor 100. In the depicted example, the bioreactor 100 has a cylindrical shape defined by a top surface 104, a bottom surface 108, and a side wall 102. For illustration purposes, the bioreactor's top and bottom surfaces 104, 108 are parallel to the x-y plane and its central axis is parallel to the z-axis on a Cartesian coordinate system. In other examples, the bioreactor 100 can have other shapes, such as a sphere, a cuboid, a cube, a prism, etc.

As shown, the drive coil 110 can include a pair of drive coil conductors 110a, 110b located on opposite sides of the cylindrical wall 102. The drive coil 110 can generate an activation magnetic field 112 that is substantially parallel to the x-y plane. In certain examples, the pair of drive coil conductors 110a, 110b can have toroidal shape so as to generate the activation magnetic field 112 in a concentrated plane defined between the pair of drive coil conductors 110a, 110b. (e.g., the drive coil conductors 110a, 110b are D-shaped). For example, FIG. 7 depicts an example configuration where the narrow beam of activation magnetic field 112 is generated by two opposing double-D coil conductors 110a, 110b placed around the circumference of the wall 102 of the bioreactor 100.

In addition, a plurality of detection coils 118 can be disposed at the bottom surface 108 of the bioreactor 100. The plurality of magnetoelastic sensors 90 can be oriented so that the return magnetic fields generated by those sensors are in parallel to the z-axis, thus being detectable by the plurality of detection coils 118. In some examples, the detection coils 118 can be disposed on the top surface 104 of the bioreactor 100. In some examples, the detection coils 118 can be disposed on both the top and bottom surfaces 104, 108 of the bioreactor 100.

In the depicted example, the plurality of magnetoelastic sensors 90 can be arranged in two or more planes (parallel to the x-y plane) inside the bioreactor 100. Thus, these magnetoelastic sensors 90 can be monitored by treating them as a stack of “2D layers” of magnetoelastic sensors 90. At a given time, the drive coil 110 can activate a selected layer of magnetoelastic sensors 90 in the bioreactor 100 and then measure their resonant responses, obtaining a 2D measurement. The 3D measurement of the bioreactor 100 can be constructed from these 2D measurements.

To activate only a layer of magnetoelastic sensors 90, the drive coil 110 can be configured to generate a narrow beam of activation magnetic field 112 (e.g., including both AC and DC magnetic field components) that only energizes the magnetoelastic sensors 90 that are within the plane of the drive coil 110. For example, when the drive coil is at z=0, only magnetoelastic sensors 90 located on the x-y plane of the bioreactor 100 at that height will be interrogated. Next, the drive coil 110 can move to another location along the z-axis, and the process can be repeated.

In the depicted example, the drive coil 110 is configured to move linearly relative to the bioreactor 100 in the direction 114 that is parallel to the z-axis. In other examples, the drive coil 110 can be stationary, whereas the bioreactor can be configured to move linearly relatively to the drive coil 110 along the z-axis. In yet alternative examples, no relative linear movement between the drive coil 110 and the bioreactor 100 is needed. Instead, a plurality of drive coils 110 can be placed along the side of the bioreactor 100 and distributed between its top and bottom surfaces 104, 108, wherein each drive coil 110 aligns with a respective 2D layer of the magnetoelastic sensors 90. Thus, by selectively activating the plurality of drive coils 110, the corresponding 2D layers of the magnetoelastic sensors 90 can be magnetically activated.

In some examples, the plurality of detection coils 118 can be arranged in a two-dimensional grid, which can be located on a plane that is perpendicular to a direction of the return magnetic field generated by the plurality of magnetoelastic sensors 90. In the example depicted in FIG. 7, a total of 24 detection coils 118 are arranged in an octagonal shape to cover the bottom surface 108 of the bioreactor 100. In other examples, different number of magnetoelastic sensors 90 can be deployed and/or arranged in different 2D patterns. For example, the number of detection coils 118 can increase with shorter inter-coil distance when a 2D layer in the bioreactor 100 has a denser array of magnetoelastic sensors 90.

Generally, the spatial resolution of a detection coil depends on the detection coil's diameter. For example, if the diameter of the detection coils 118 is 1.75 cm, then the default spatial resolution for the detection coils 118 along the x- and y-axes are both 1.75 cm. In some examples, the x and y spatial resolutions of the detection coils 118 can be improved by performing multiple measurements after slightly moving the detection coils 118 along these two directions. A distance of positional shift along a shifting axis (e.g., x- or y-axis) is smaller than a distance between two adjacent detection coils 118 along the shifting axis. For example, if four measurements are made by moving along x-axis and then y-axis by 8.75 mm each, the spatial resolution of the detection coils 118 can be improved from 1.75 cm to 8.75 mm. In other words, by slightly shifting position in the x-y plane relative to the bioreactor 100 and performing multiple measurements in both the original and shifted positions, higher spatial resolution can be achieved by the detection coils 118.

In an alternative example, the number of detection coils 118 can be much smaller than the number of magnetoelastic sensors 90 in a 2D layer (e.g., in some examples, there can be only detection coil 118). To detect the return magnetic fields generated by a plurality of magnetoelastic sensors 90 in a 2D layer, the detection coil(s) 118 can be configured to move around (e.g., shifting position in the x-y plane so that it can sequentially pick up the return magnetic field signal from each of the magnetoelastic sensors 90).

As described herein, the activation magnetic field (e.g., 84 or 112) generated by the drive coils (e.g., 82 or 110) is restricted to relatively low-gradient (e.g., <1 GT/m) to reduce the effect of magnetic field on cell behaviors. In some examples, the activation magnetic field can be constrained to be less than 0.1 T with a gradient <10 T/m.

In addition, as described herein, the drive coils (e.g., 82 or 110) can be configured to have a miniaturized profile while reducing localized heating. For example, in certain examples, heatsinks can be installed at the back of a drive coil to accelerate heat dissipation.

### Example Monitoring System

FIG. 8 schematically illustrates an example magnetoelastic sensor monitoring system. As shown, a magnetoelastic sensor 120 (or magnetoelastic microcarrier) can be placed within a magnetic coil system 122, which include a DC coil and an AC coil, as described above. The DC coil can be connected to a DC power supply 124 to generate a DC magnetic field component to maximize the magnetoelastic sensor's resonance signal. The AC coil can be connected to a control unit 126 which includes a drive circuit configured to provide an AC drive current to the AC coil and a detection circuit configured to detect and process the return magnetic field signal.

In certain examples, the magnetic coil system 122 can include a dedicated detection coil configured to detect the return magnetic field signal generated by the magnetoelastic sensor 120. In other examples, the AC coil can be used both as a drive coil to generate the activation magnetic field and as a detection coil to pick up the return magnetic field generated by the magnetoelastic sensor 120. In latter circumstances, the control unit 126 can include switches and/or buffers which separate the drive circuit from the detection circuit.

In some examples, the control unit 126 can also be configured to measure an impedance based on the return magnetic field signal. The return magnetic field signal (which can include the measured impedance) can be sent to a computer system 128 (as described further below) for further analysis, e.g., to calculate one or more resonant response parameters of the magnetoelastic sensor 120.

FIG. 9 is a block diagram illustrating a monitoring system configured for longitudinal monitoring of cell growth in a bioreactor, according to one example. As shown, a 3D bioreactor 130 can include a plurality of magnetoelastic microcarriers 140 configured to support cell attachment and growth. The magnetoelastic microcarriers 140 can be arranged in one or more layers, as described above.

A drive coil 132 can be placed outside the bioreactor 130 and connected to a drive circuit 142. Similar to 110, the drive coil 132 can be configured to be movable relative to the bioreactor 130 such that the magnetoelastic microcarriers 140 at different layers can be selectively activated by the activation magnetic field 134 generated by the drive coil 132, as described above. Likewise, an array of detection coils 138 (similar to 118) can be disposed on one side of the bioreactor 130 and configured to detect the return magnetic fields 136 generated by the magnetoelastic microcarriers.

The drive circuit 142 can be configured to generate an activation signal 144 (also referred to as the “excitation signal”) to the drive coil 132. The activation signal 144 can include an AC drive current with variable frequencies (e.g., up to 50 MHz or higher) to generate the corresponding AC magnetic field component to activate the magnetoelastic microcarriers 140. The drive circuit 142 can also be configured to provide a DC biasing current to the drive coil 132 to maximize the resonant response of the magnetoelastic microcarriers 140.

As described further below, the drive circuit 142 can be configured to alternate between an ON phase and an OFF phase. In the ON phase, the drive circuit 142 can be configured to generate the AC drive current for a plurality of drive cycles, and in the OFF phase, the drive circuit 142 can be configured to generates no AC drive current. In some examples, the duration of the OFF phase can be determined based on a number of detection coils 138 in the system.

In some examples, when the magnetoelastic microcarriers 140 have multiple resonant frequencies f₁,f₂,f₃, etc. (e.g., when the embedded magnetoelastic sensors have different lengths), the drive circuit 142 can be configured to generate the activation signal 144 comprising the multiple resonant frequencies f₁,f₂,f₃, etc. For example, the activation signal 144 can be composed of multiple sinewaves at frequencies of the expected resonant frequencies (e.g., f₁,f₂,f₃, etc.) of the magnetoelastic microcarriers 140.

In some examples, the array of detection coils 138 can be connected to a multiplexer 146 so that the return magnetic field signals 145 detected by the detection coils 138 can be sequentially processed by a single detection circuit 148.

As described herein, the detection circuit 148 can be configured to amplify, filter (e.g., for noise removal) and digitize (e.g., through and analog-to-digital converter, or ADC) the return magnetic field signals 145. In some examples, the detection circuit 148 can also be configured to extract multiple frequency components of the return magnetic field signals 145, as described further below. As such, the detection circuit 148 can also be referred to as a signal processing unit.

Both the drive circuit 142 and the detection circuit 148 can be connected to and controlled by a processing unit 150, which can be a microcontroller or a central processing unit (CPU) inside a computer or computer system. For example, the processing unit 150 can run a software application which controls the frequencies of the activation signal 144, the movement of the drive coil 132 relative to the bioreactor 130, the movement of the detection coils 138 (e.g., positional shift to increase the spatial resolution), the amplification and filter setting within the detection circuit 148, the sampling frequency of the ADC, etc.

As described herein, the processing unit 150 can be configured to analyze the return magnetic field signals 145 to calculate one or more resonant response parameters of the corresponding magnetoelastic microcarriers 140. In certain examples, the processing unit can be configured to determine an origin of the return magnetic field detected by the array of detection coils 138. For example, because each detection coil 138 is more sensitive to closer magnetoelastic microcarriers, the origin of the returned magnetoelastic field signal can be determined by comparing the measured fields among the detection coils 138, and finding a maximum signal strength of the detected return magnetic field among the array of detection coils 138.

In certain examples, the one or more resonant response parameters calculated by the processing unit 150 can include a resonant frequency of the magnetoelastic sensor (or magnetoelastic microcarrier), a phase angle of impedance at the resonant frequency, a magnitude of impedance at the resonant frequency, a resonance quality factor, etc. In certain examples, the processing unit 150 can be configured to detect a shift of the resonant frequency measured at two different points in time, a shift of the phase angle of impedance measured at two different points in time, a shift of the magnitude of impedance measured at two different points in time, or a shift of the resonance quality factor measured at two different points in time.

In certain examples, to measure a resonant frequency of a magnetoelastic sensor embedded in a microcarrier, a frequency sweep can be conducted by sequentially activating the drive coil with a number of activation frequencies within a spectrum containing the magnetoelastic sensor's characteristic resonant frequency and measuring the frequency response of the corresponding return magnetic field signal. For example, the frequency spectrum can cover both the ascending and descending limbs of the magnetoelastic sensor's expected resonant frequency. Finer resolution of resonant frequency measurement can be achieved by decreasing a step size (i.e., difference) between adjacent activation frequencies in the spectrum. In certain examples, the speed of frequency sweep can be increased by reducing the number of activation frequencies used in the frequency sweep, while interpolation technique (e.g., linear interpolation) can be used to estimate the frequency response between two adjacent activation frequencies.

As described herein, the resonance quality factor refers to a dimensionless parameter that describes how underdamped an oscillator or resonator is. In certain examples, the resonance quality factor can be a Q factor, which measures a resonant frequency-to-bandwidth ratio of the resonator. For example, the resonance quality factor of a magnetoelastic sensor can be defined as a ratio of the sensor's resonant frequency to its resonance width or full width at half maximum, i.e., the bandwidth over which the power of vibration is greater than half the power at the resonant frequency.

In certain examples, the processing unit 150 can access a computer readable media (as described further below), which can store one or more calibration curves associated with the magnetoelastic microsensors embedded in the microcarriers 140. Based on the calculated one or more resonant parameters and the one or more calibration curves, the processing unit 150 can generate one or more metrics indicating the cell growth condition within the bioreactor 130. For example, the one or more metrics can include counts of cells attached to the magnetoelastic microcarriers 140, the local pH values surrounding the magnetoelastic microcarriers 140, the local concentrations of a specific analyte or biomarker surrounding the magnetoelastic microcarriers 140, etc. The processing unit 150 can track a variation of the one or more resonant response parameters over a period of time so as to accomplish for longitudinal monitoring of the cell growth in the bioreactor 130.

In alternative examples, the multiplexer 146 can be optional. Instead, parallel processing technique can be used to simultaneously process the return magnetic field signals 145 detected by the array of detection coils 138. For example, each detection coil 138 can be connected to a dedicated detection circuit (similar to 148), which in turn is connected to a dedicated microprocessor or controller (similar to 150).

### Example Method of Bioreactor Scanning

A specific example of scanning the plurality of magnetoelastic microcarriers 140 inside the bioreactor 130 is described herein. It is to be understood that specific parameters described below are merely illustrative, and different parameters can be used depending on factors such as the number of magnetoelastic microcarriers, number of scanning layers, number of detection coils, etc.

As noted above, to measure the resonant frequencies of multiple magnetoelastic microcarriers 140, the drive circuit 142 can first generate an activation signal 144 containing multiple sinewaves at frequencies of the expected resonances of the magnetoelastic microcarriers 140. The activation signal can be configured to last for about a predefined number of cycles (e.g., 100) cycles (i.e., ON phase) to provide the activation magnetic field 134 to vibrate the magnetoelastic microcarriers 140 (e.g., for 20 MHz resonant frequency, 100 cycles last for about 5 μs). The drive circuit 142 can then be turned off (i.e., OFF phase), causing the magnetoelastic microcarriers 140 to experience exponentially decaying vibrations at their resonant frequencies for a short period of time (e.g., 10-100 cycles depending on the mechanical damping). The decaying vibrations from multiple magnetoelastic microcarriers 140, in the absence of the activation magnetic field 134, can be measured by the detection circuit 148 as induced voltages across the detection coils 138. The resonant frequency of an individual magnetoelastic microcarrier can be determined by first digitally filtering out the frequency components from other magnetoelastic microcarriers (as described further below) and then measuring the frequency of the decaying vibration. Similarly, the resonance quality of a magnetoelastic microcarrier can be calculated, e.g., by measuring the duration of the decaying vibration signal (after digital filtering). The scanning speed can be affected by the number of detection coils. For example, assuming 5 μs of ON phase or energizing period and 5 μs of OFF phase for measurement, the signal acquisition for 24 detection coils can take about 240 μs, which represents the duration to perform a single measurement on a single layer of magnetoelastic microcarriers 140. The volume of the bioreactor 130 can be interrogated by moving the drive coil 132 to align with different layers and then repeat the measurement process.

### Example Signal Processing Circuit and Method

In some examples, signal processing techniques can be used in conjunction with the drive circuit (e.g., 142) and the detection circuit (e.g., 148) to simultaneously monitor a plurality of magnetoelastic sensors having different characteristic resonant frequencies. Specifically, a drive coil can be configured to generate an activation magnetic field having a frequency spectrum defined by (or covering) a plurality of resonant frequencies respectively corresponding to the plurality of magnetoelastic sensors. The plurality of magnetic sensors can then vibrate and generate their respective return magnetic fields. A detection coil can pick up the combined return magnetic field signals generated by the plurality of magnetoelastic sensors. Certain signal processing techniques, as described further below, can be applied to separate and determine the frequency responses corresponding to the plurality of magnetoelastic sensors.

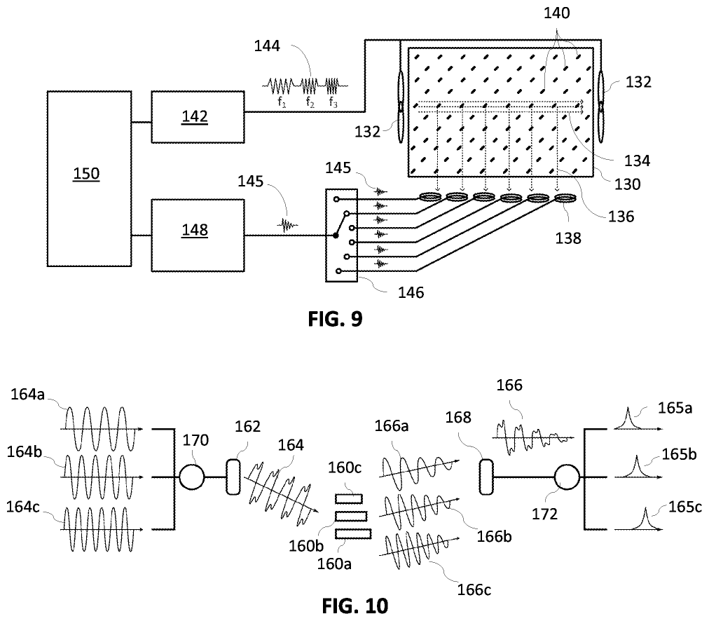

FIG. 10 schematically illustrates the concept of synthesizing a multi-frequency activation signal to drive multiple magnetoelastic sensors and extracting each sensor's frequency response from a mixed signal containing return magnetic field signals generated by all magnetoelastic sensors.

As shown, the magnetoelastic sensors 160a, 160b, and 106c have different lengths, thus having different characteristic resonant frequencies. Sinusoidal signals 164a, 164b, and 164c of different frequencies, close or at the expected resonant frequencies of the magnetoelastic sensors 160a, 160b, and 160c, can be generated. In some examples, the frequencies of the signals 164a, 164b, and 164c do not have to match exactly the corresponding resonant frequencies of magnetoelastic sensors as long as they are about 10% within the corresponding resonant frequencies. These signals (e.g., 164a, 164b, and 164c) can be combined with a circuitry 170, and the combined signal can be sent to a drive coil 162 to generate an activation magnetic field 164 that combines the frequency spectra of the signals 164a, 164b, and 164c.

The magnetoelastic sensors 160a, 160b, and 160c can be excited by the activation magnetic field 164 and vibrate. When the activation magnetic field 164 is turned off, the magneto-mechanical energy absorbed by these magnetoelastic sensors 160a, 160b, and 160c will be released as exponentially decaying sinewaves (also referred to as “ring-downs”), generating corresponding return magnetic field signals (e.g., 160a, 160b, 160c). Each magnetoelastic sensor can generate its own unique ring-down and the return magnetic field signal at its characteristic resonant frequency.

A detection coil 168 can capture the return magnetic field signals (e.g., 160a, 160b, 160c) from the magnetoelastic sensors (e.g., 160a, 160b, 160c) that are within its detection region as a combined time-domain waveform 166. The combined waveform 166 can be processed, e.g., through a detection circuit 172, to generate the frequency responses (e.g., 165a, 165b, 165c) corresponding to respective magnetoelastic sensors (e.g., 160a, 160b, 160c).

For example, as described further below, the combined waveform 166 can be digitized and filtered with multiple digital band-pass (BP) filters, wherein each filter has a passband centered on and covering the expected resonant frequency range of an individual magnetoelastic sensors (e.g., 160a, 160b, 160c). The BP filtered signal can be converted to frequency-domain, e.g., via Fourier transformation, and generate a frequency-domain resonance plot. The resonant frequency and other resonance response parameters (e.g., resonance quality factor, impedance amplitude, phase, etc.) can be determined from the frequency-domain resonance plot. In an alternative example, a moving BP filter can scan through the frequency range that covers all the magnetoelastic sensors (e.g., 160a, 160b, and 160c) and determine the frequencies that correspond to local peak signals. These frequencies can be deemed to be the resonant frequencies of the corresponding magnetoelastic sensors.

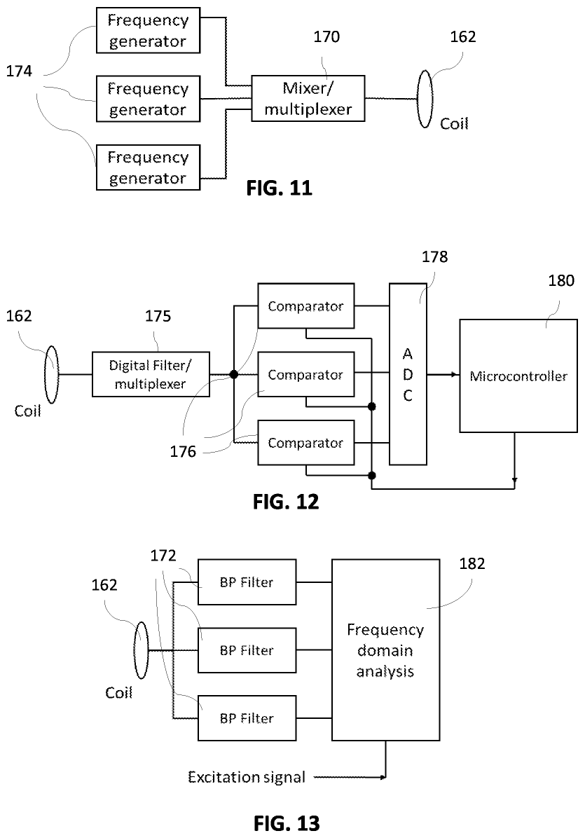

FIG. 11 is a block diagram of an example circuit for generating a multi-frequency activation signal to drive multiple magnetoelastic sensors. Because the excitation of a magnetoelastic sensor is typical carried out over a frequency spectrum around its characteristic resonant frequency, simultaneous excitation of multiple magnetoelastic sensors can be accomplished through a composite signal having a frequency spectrum covering (or comprising) all their characteristic resonant frequencies. As shown, a plurality of frequency generators 174 can be used to generate sinusoidal signals (e.g., 164a, 164b, 164c) of different frequencies. Examples of frequency generators 174 include direct digital synthesis (DDS), oscillators, and programmable timer circuits, etc. The generated signals (e.g., 164a, 164b, 164c) can be combined using the circuitry 170 (e.g., frequency mixers or frequency domain multiplexers) for combining the signals, to generate a composite signal that combines the frequency spectra of the original signals (e.g., 164a, 164b, and 164c). The composite signal can modulate the AC drive current and activate the drive coil 162.

As described herein, time- and/or frequency-domain techniques can be used to separate and determine different frequency spectrum characteristic of each magnetoelastic sensor from the combined waveform (e.g., 166) detected by the detection coil 168. As noted above, in certain examples, the detection coil 168 and the drive coil 162 can be the same so long as there are switches/buffers isolating the drive circuitry from the detection circuitry.

FIG. 12 is a block diagram of an example circuit for extracting frequency responses of multiple magnetoelastic sensors from a mixed return magnetic field signal based on time-domain analysis. As shown, the circuit can include one or more comparators 176, which can be inverting comparators, non-inverting comparators, and/or their combinations. The comparators 176 generate pulses based on free vibration of the magnetoelastic sensors. The comparators 176 can also be supplied with reference voltage signals. The pulse generated from the comparators 176 can then be analyzed by a microcontroller 180 (or computer) either directly or with additional circuitries including filters, digital signal processing (DSP) microcontrollers, and/or ADCs. In certain examples, the analysis with comparators 176 can also be carried out after separating different frequencies using digital filters and/or multiplexers 175. For example, the resonant frequency of the magnetoelastic sensor can be deter mined by taking the ratio of the number of pulses generated by the comparators for a given tune period. The Q factor can be determined by taking the difference in the total number of pulses generated by two comparators—e.g., one with high reference voltage and one with low reference voltage.

FIG. 13 is a block diagram of an example circuit for extracting frequency responses of multiple magnetoelastic sensors from a mixed return magnetic field signal based on frequency-domain analysis. Frequency domain analysis for multiple magnetoelastic sensors can be an alternative to the time-domain analysis described above. This approach can involve frequency spectrum analysis based on the excitation signal (e.g., the composite signal synthesized from signals 164a, 164b, and 164c). The characteristics of the return magnetic field signal can be analyzed by a frequency-domain analyzer 182 for change in power, phase, and/or magnitude with respect to the excitation signal. In certain examples, the phase and/or impedance can be measured by using network analyzers, spectrum analyzers, DSP microcontrollers, impedance measurement circuitry, and/or software-based detection along with ADCs and/or coder-decoders (CODECs).

In certain examples, hardware- and/or software-based BP filters 175 can be applied to the combined time-domain waveform (e.g., 166) to isolate respective frequency spectra corresponding to different magnetoelastic sensors. Such filtering can be followed by the use of time-domain or frequency-domain based analysis technique described above. Examples implementations include using a circuitry for signal processing filtering frequencies in different combination, ADC/CODEC based signal detection followed by software-based signal processing and DSP microcontrollers for processing.

### Example Experimental Results

Example experimental results are described below to illustrate the application of magnetoelastic sensors to track the cell growth. Although in the described experiment, specific cells with specific seeding densities were cultured in a specific bioreactor having specific magnetoelastic sensors, it should be understood that the same principles can be applied to other cell types with different seeding densities, other types of bioreactors, and other types of magnetoelastic sensors. It should also be understood that the same principles described herein can be used to monitor the local microenvironment of a cell culture in a bioreactor.

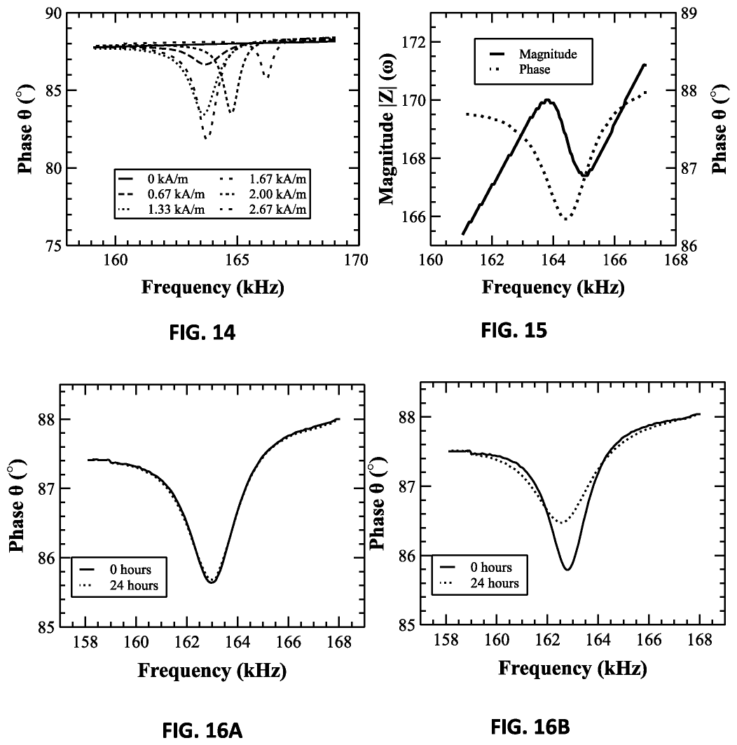

In the experiment, custom fabricated magnetoelastic sensors are used to monitor the attachment of anchorage-dependent mammalian cells in 2D in vitro cell cultures. A calibration curve for the magnetoelastic sensors was obtained and shown in FIG. 5. The magnetoelastic sensors are disposed inside a chamber slide that is placed inside a custom monitoring system similar to the one depicted in FIG. 8. The monitoring system can generate a DC biasing field to increase the magnetoelastic effect of the magnetoelastic sensors, leading to larger resonance. By varying the magnitude of the DC biasing field, the largest change in sensor response (e.g., the phase angle at the resonant frequency θ) was observed at 1.67 kA/m field, as shown in FIG. 14. Thus, this DC biasing field can be selected for all sensor measurements in the experiment.

FIG. 15 shows that at this optimal biasing field, the resonant frequency of the magnetoelastic sensors occurred at about 164.4 kHz. Note that in this experiment, the phase of impedance at resonant frequency, θ, was used to track cell attachment to the magnetoelastic sensors. FIG. 15 also shows the magnitude of impedance curve, which has a relatively steep slope around the resonant frequency. In some examples, the magnitude of impedance at the resonant frequency can also be used to track cell attachment to the magnetoelastic sensors, for example, by first removing the steep-sloped component corresponding to a background signal. To do so, for example, a background signal, measured without the presence of magnetoelastic sensors, can be first taken and stored prior to measurement. A background calibration routine can then be performed by subtracting the measured frequency spectrum (with the presence of magnetoelastic sensors) to this background signal. Note that only one background signal is needed at the beginning of the experiment.

In the experiment, L929 fibroblast cells were seeded at varying densities ranging from 5×10³ cells/cm' to 4×10⁴ cells/cm'. Magnetoelastic sensor measurements were taken at two different time points, before (0 hr.) and after cell attachment (24 hrs.) to the sensor surface. As shown in FIG. 16A, no observable change in the measured phase at resonant frequency θ when the magnetoelastic sensors were placed in media without cells after 24 hours, indicating adsorption of proteins from the serum containing media did not affect the sensor readout. In contrast, as shown in FIG. 16B, when the magnetoelastic sensors were placed in media with cells (e.g., with seeding density 4×10⁴ cells/cm), a significant change in the sensor response was observed after 24 hours due to cell attachment to the magnetoelastic sensors.

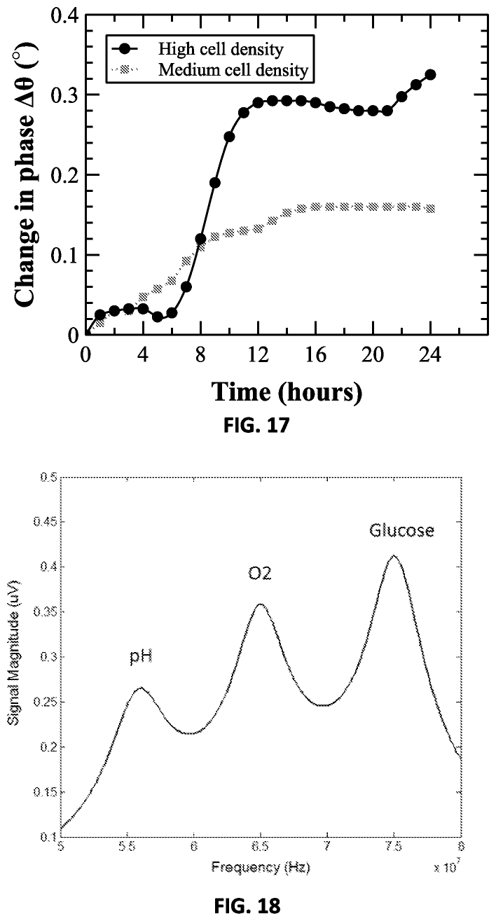

In the experiment, the calibrated magnetoelastic sensors (see e.g., FIG. 5) were used to monitor the growth of L929 fibroblast cells in real time, as shown in FIG. 17. This was done through two separate configurations using high and medium cell seeding densities on the surface of magnetoelastic sensors. As expected, sensor response with high cell seeding numbers (4×10⁴ cells/cm') is shown to capture different stages of cell attachment and proliferation. For example, a small change in phase was observed during the initial stages after cell seeding (0-4 hours), whereas there was a sharp increase in sensor response in the next 4-8 hours corresponding to the expected period for cells to settle and attach over the sensor surface. These results were expected as cell attachment to a surface is a dynamic process that consists of initial period of cells settling down on the surface, followed by cell adhesion to extracellular matrices involving formation of numerous receptor mediated binding complexes. These cell-to-surface interactions via binding to extracellular matrix act in coordination to strengthen the mechanical anchorage of cells over time, resulting in enhanced force distribution among bound receptors through local membrane stiffening. In case of the magnetoelastic sensors, the settlement of cells and progressive strengthening of the mechanical anchorage on sensor surface led to the initial rise in the sensor response as show in FIG. 17. Following cell attachment to the surface, sensor response plateaued and gradually increased again at 24 hours due to cell proliferation. Similar pattern of sensor response was observed for medium cell seeding density (2×10⁴ cells/cm²), as shown in FIG. 17. However, the magnitude of phase change was lower than high seeding density, which was expected as the number of cells attached on the sensor surface for medium seeding density was lower.

### Example Simulation Results

Example simulation results are described below to illustrate the application of magnetoelastic sensors to simultaneously measure local concentrations of multiple analytes in a bioreactor. Although in the described experiment, specific analytes were detected in a specific simulation setup, it should be understood that the same principles can be applied to for the detection of other analytes in different measurement setups.

In the simulation, a plurality of magnetoelastic microcarriers are disposed inside a bioreactor. Each magnetoelastic microcarrier contains three individual magnetoelastic sensors having different lengths, e.g., 30 μm for Sensor C, 34 μm for Sensor B, and 39 μm for Sensor A, respectively. Sensors A, B and C were respectively coated with pH, oxygen, and glucose sensing coatings. The resonant spectrum of the three magnetoelastic sensors was calculated and plotted in FIG. 18. As shown, the pH sensor (with the longest length) has the lowest resonant frequency and the glucose sensor (with the shortest length) has the highest resonant frequency. There are some overlaps among the resonant curves from the three magnetoelastic sensors, but the resonant peak for each sensor is well defined.

In the simulation, the bioreactor was a cube with side lengths of 5 cm. The magnetoelastic microcarriers (each contains three individual magnetoelastic sensors for detecting pH, oxygen, and glucose) were assumed to be uniformly distributed inside the medium at a spacing of 1 mm from all sides. The drive coil, placed on the y-z plane at the origin of a Cartesian coordinate, was a circular coil array that can generate a highly concentrated activation magnetic field with the half-power values occurs within ±5 mm from the center of the drive coil. The detection coil was a circular loop (2 mm in diameter) at the side of the bioreactor. Due to the symmetry of the bioreactor, only simulation results on the x-y plane (i.e., 2D) are shown. A 3D simulation would be similar to the 2D simulation but with iterations along the z-direction.

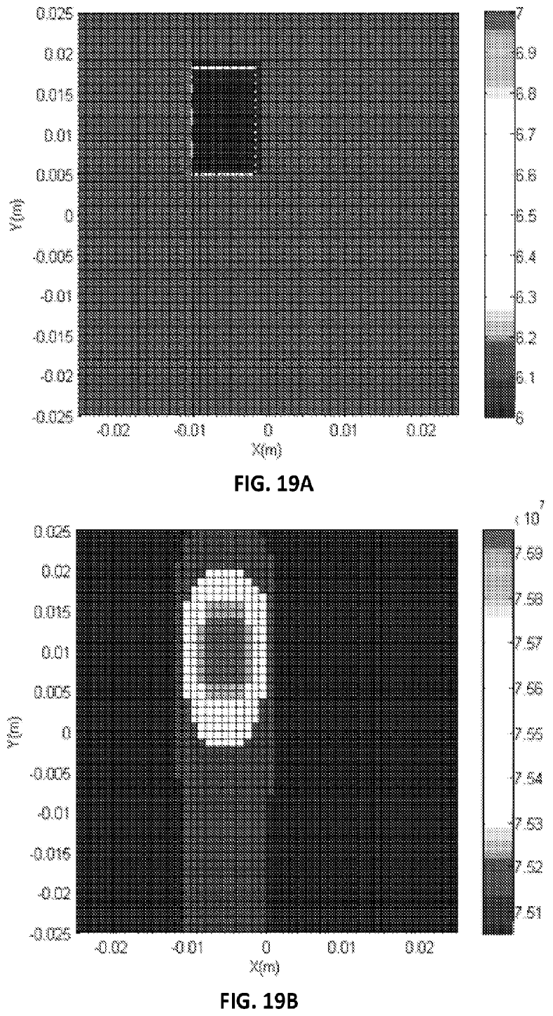

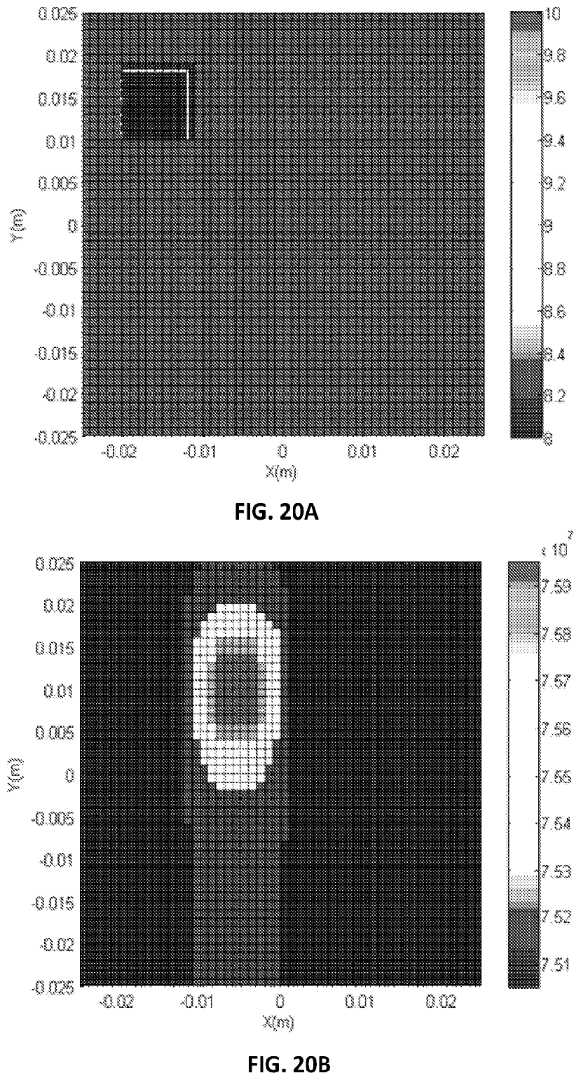

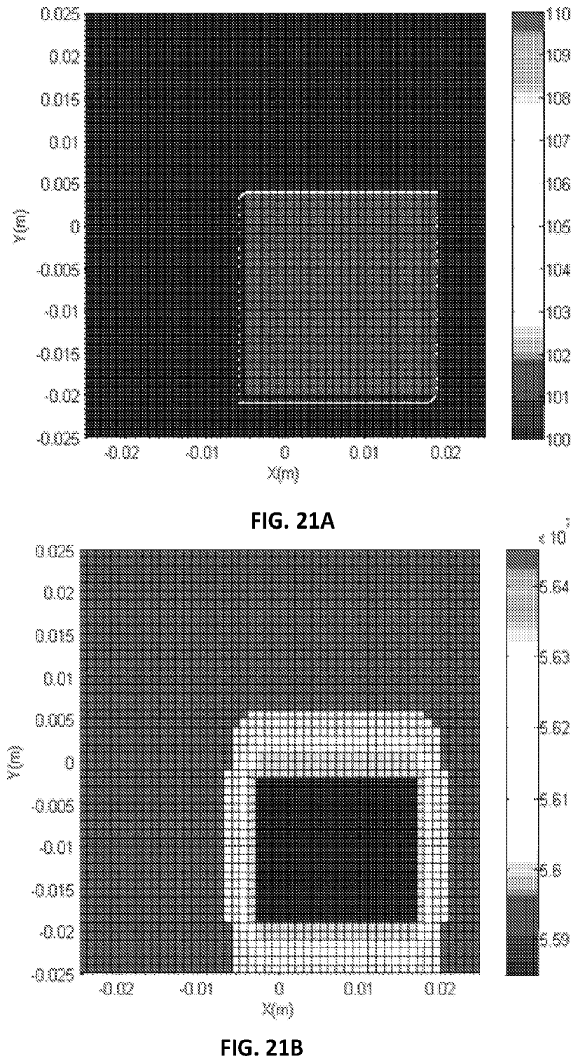

In the simulation, the pH value was set to be 7 throughout the whole x-z plane, except for a section of 10 mm×20 mm that was pH 6 (see FIG. 19A). Similarly, the oxygen value was set to 10 except for a section of 10 mm×10 mm that was 8 (see FIG. 20A), and the glucose value was set to 100 except for a section of 25 mm×25 mm that was 120 (see FIG. 21A). Theoretical simulation was programmed in Matlab. When coil was at x=0, the program first calculated the magnetic field at magnetoelastic sensors at all locations, and then calculated the magnetic fields captured by the detection coil. The process was repeated again after setting x=1 mm for the coil location, and so on until the responses from all magnetoelastic sensors were measured. The resonant frequencies of all three types of magnetoelastic sensors were calculated by determining the frequencies that correspond to the highest peaks (see FIG. 18).

If a drive coil can provide a highly focused magnetic field that can pinpoint to a specific magnetoelastic sensor, the received signal will be “clean” in the sense that no other magnetoelastic sensors will contribute to the measurements. However, since the activation magnetic field is like a thin wall that moves through the sample volume, the received return magnetic field signal from a particular magnetoelastic sensor can be affected by its surrounding neighbors.

FIG. 19B plots the resonant frequencies of Sensor A (pH sensor) in magnetoelastic sensors throughout the bioreactor, captured by the detection coil. The results show that the resonant frequencies of Sensors A were higher at regions where the pH value was lower, as expected. It is also noted that the pattern of the measurements (FIG. 19B) are not rectangular shape like the pH map (FIG. 19A), but contains noises at boundaries where the pH value changes. This is due to the interferences from neighboring magnetoelastic sensors. Similar patterns can be observed for the oxygen and glucose measurements (see FIGS. 20B and 21B).

In certain examples, the neighboring interferences shown in FIGS. 19B, 20B and 21B can be reduced, for example, by reducing the beam size of the activation magnetic field (i.e., make the drive field more focused). Further, in the simulation, where the parameters of interest have erupt changes in terms of concentration (e.g., from pH 7 to pH 6 instead of a gradual change) and dimension (e.g., pure rectangular), neighboring interferences can be amplified due to rapid change between the parameter values at two adjacent magnetoelastic sensors. In practice, the changes in concentrations are gradual, so the interferences generally do not skew the measurements as much.

### Example Magnetoelastic Stimulation

In certain examples, the magnetoelastic sensors (or magnetoelastic microcarriers) described above can also be used as actuators to modulate cell growth. For example, in certain circumstances, the mechanical vibrations of the magnetoelastic microcarriers when exposed to the activation magnetic field can affect growth and activity of cells attached to the microcarriers. Thus, by using the magnetoelastic microcarriers as both sensors and actuators, it is not only feasible to longitudinally monitor cell growth in a bioreactor as described above, but also feasible to optimize cell growth via precise mechanical stimulations delivered by the magnetoelastic microcarriers, e.g., by modulating the amplitude, frequency, and/or duration of the mechanical vibrations of the magnetoelastic microcarriers. In certain examples, to apply the magnetoelastic microcarriers to modulate cells, the vibration amplitude can be set to be at least 10 times larger than that during the sensing operation. This can be accomplished, for example, by increasing the excitation magnetic field during the modulation operation and/or reducing the field strength during the measurement mode.

### Example Computing Systems

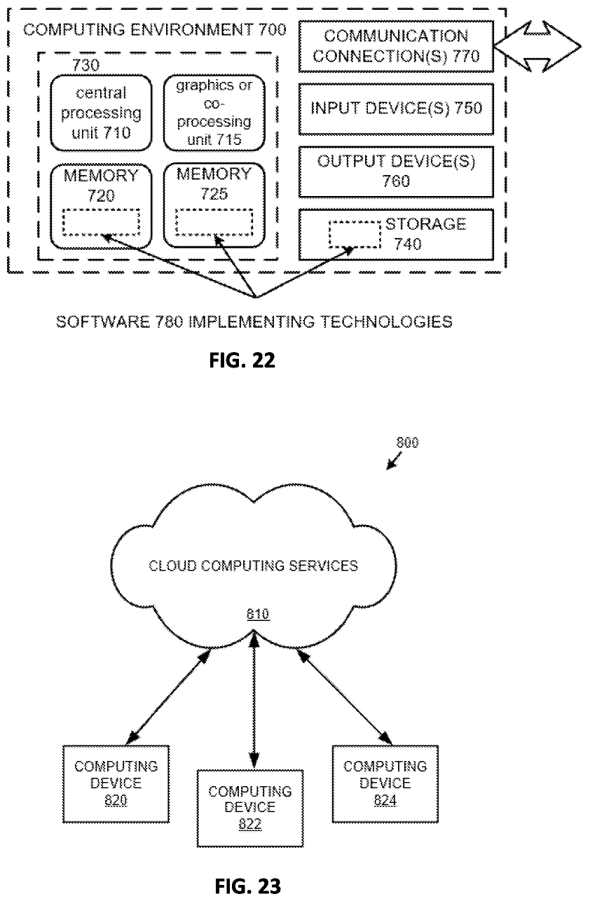

FIG. 22 depicts an example of a suitable computing system 700 in which the described innovations can be implemented. For example, the computing system 700 can be used as the computing system 128 depicted in FIG. 8, the processing unit 150 depicted in FIG. 9, and/or the microcontroller 180 depicted in FIG. 12. The computing system 700 is not intended to suggest any limitation as to scope of use or functionality of the present disclosure, as the innovations can be implemented in diverse computing systems.

With reference to FIG. 22, the computing system 700 includes one or more processing units 710, 715 and memory 720, 725. In FIG. 22, this basic configuration 730 is included within a dashed line. The processing units 710, 715 execute computer-executable instructions, such as for implementing the features described in the examples herein. A processing unit can be a general-purpose central processing unit (CPU), processor in an application-specific integrated circuit (ASIC), or any other type of processor. In a multi-processing system, multiple processing units execute computer-executable instructions to increase processing power. For example, FIG. 22 shows a central processing unit 710 as well as a graphics processing unit or co-processing unit 715. The tangible memory 720, 725 can be volatile memory (e.g., registers, cache, RAM), non-volatile memory (e.g., ROM, EEPROM, flash memory, etc.), or some combination of the two, accessible by the processing unit(s) 710, 715. The memory 720, 725 stores software Error! Reference source not found.80 implementing one or more innovations described herein, in the form of computer-executable instructions suitable for execution by the processing unit(s) 710, 715.

A computing system 700 can have additional features. For example, the computing system 700 includes storage 740, one or more input devices 750, one or more output devices 760, and one or more communication connections 770, including input devices, output devices, and communication connections for interacting with a user. An interconnection mechanism (not shown) such as a bus, controller, or network interconnects the components of the computing system 700. Typically, operating system software (not shown) provides an operating environment for other software executing in the computing system 700, and coordinates activities of the components of the computing system 700.

The tangible storage 740 can be removable or non-removable, and includes magnetic disks, magnetic tapes or cassettes, CD-ROMs, DVDs, or any other medium which can be used to store information in a non-transitory way and which can be accessed within the computing system 700. The storage 740 stores instructions for the software implementing one or more innovations described herein.

The input device(s) 750 can be an input device such as a keyboard, mouse, pen, or trackball, a voice input device, a scanning device, touch device (e.g., touchpad, display, or the like) or another device that provides input to the computing system 700. The output device(s) 760 can be a display, printer, speaker, CD-writer, or another device that provides output from the computing system 700.

The communication connection(s) 770 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

The innovations can be described in the context of computer-executable instructions, such as those included in program modules, being executed in a computing system on a target real or virtual processor (e.g., which is ultimately executed on one or more hardware processors). Generally, program modules or components include routines, programs, libraries, objects, classes, components, data structures, etc. that perform particular tasks or implement particular abstract data types. The functionality of the program modules can be combined or split between program modules as desired in various examples. Computer-executable instructions for program modules can be executed within a local or distributed computing system.

For the sake of presentation, the detailed description uses terms like “determine” and “use” to describe computer operations in a computing system. These terms are high-level descriptions for operations performed by a computer, and should not be confused with acts performed by a human being. The actual computer operations corresponding to these terms vary depending on implementation.

### Example Computer-Readable Media

Any of the computer-readable media herein can be non-transitory (e.g., volatile memory such as DRAM or SRAM, nonvolatile memory such as magnetic storage, can be implemented by storing in one or more computer-readable media (e.g., computer-readable storage media or other tangible media). Any of the things (e.g., data created and used during implementation) described as stored can be stored in one or more computer-readable media (e.g., computer-readable storage media or other tangible media). Computer-readable media can be limited to implementations not consisting of a signal.

Any of the methods described herein can be implemented by computer-executable instructions in (e.g., stored on, encoded on, or the like) one or more computer-readable media (e.g., computer-readable storage media or other tangible media) or one or more computer-readable storage devices (e.g., memory, magnetic storage, optical storage, or the like). Such instructions can cause a computing device to perform the method. The technologies described herein can be implemented in a variety of programming languages.

### Example Cloud Computing Environment

FIG. 23 depicts an example cloud computing environment 800 in which the described technologies can be implemented, including, e.g., the system disclosed above and other systems herein. In certain examples, the cloud computing environment 800 can be used to allow remote control and monitoring of bioreactors described herein. For example, through the cloud computing environment, an operator can remotely program the frequency, amplitude, and/or other parameters that control the activation magnetic field, and/or remotely monitor the cell growth in a reactor.

The cloud computing environment 800 comprises cloud computing services 810. The cloud computing services 810 can comprise various types of cloud computing resources, such as computer servers, data storage repositories, networking resources, etc. The cloud computing services 810 can be centrally located (e.g., provided by a data center of a business or organization) or distributed (e.g., provided by various computing resources located at different locations, such as different data centers and/or located in different cities or countries).

The cloud computing services 810 are utilized by various types of computing devices (e.g., client computing devices), such as computing devices 820, 822, and 823. For example, the computing devices (e.g., 820, 822, and 824) can be computers (e.g., desktop or laptop computers), mobile devices (e.g., tablet computers or smart phones), or other types of computing devices. For example, the computing devices (e.g., 820, Error! Reference source not found.22, and Error! Reference source not found.24) can utilize the cloud computing services 810 to perform computing operations (e.g., data processing, data storage, and the like).

In practice, cloud-based, on-premises-based, or hybrid scenarios can be supported.

### Example Implementations

Although the operations of some of the disclosed methods are described in a particular, sequential order for convenient presentation, such manner of description encompasses rearrangement, unless a particular ordering is required by specific language set forth herein. For example, operations described sequentially can in some cases be rearranged or performed concurrently.

### Example Embodiments

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application. Any of the following examples can be implemented.
Example 1. A microcarrier comprising:

a housing configured for cells to attach thereto and grow; and

a magnetoelastic sensor placed inside the housing;

wherein the magnetoelastic sensor is configured to vibrate with a specific resonant response when exposed to a first magnetic field and generate a second magnetic field.
Example 2. The microcarrier of example 1, wherein the housing is porous.
Example 3. The microcarrier of any one of examples 1-2, wherein the magnetoelastic sensor is one of a plurality of magnetoelastic sensors, wherein the plurality of magnetoelastic sensors have different lengths so that they vibrate with different resonant responses when they are exposed to the first magnet field.
Example 4. The microcarrier of any one of examples 1-3, wherein the magnetoelastic sensor comprises a magnetoelastic substrate, wherein the magnetoelastic substrate has a rod or a rectangular strip shape.
Example 5. The microcarrier of example 4, wherein the magnetoelastic substrate has an aspect ratio between about 2 and about 4, wherein the aspect ratio is calculated as a ratio of an axial length of the magnetoelastic substrate to a maximum width in a cross-section that is perpendicular to the axial length of the magnetoelastic substrate.
Example 6. The microcarrier of any one of examples 4-5, wherein the magnetoelastic sensor comprises a first coated layer encapsulating the magnetoelastic substrate, wherein the first coated layer comprises a biocompatible material.
Example 7. The microcarrier of example 6, wherein the magnetoelastic sensor comprises a second coated layer encapsulating the first coated layer, wherein the second coated layer is configured to promote attachment of cells to the housing without destructing the cells.
Example 8. The microcarrier of example 7, wherein the magnetoelastic sensor comprises a third coated layer, wherein the third coated layer comprises a chemical configured to react to an analyte surrounding the microcarrier such that reaction of the chemical to the analyte causes shrink or swell of the third coated layer, thereby changing the magnetoelastic sensor's resonant response.
Example 9. A bioreactor comprising:

a chamber configured to grow cells; and

a microcarrier disposed inside the chamber and configured for cells to attach thereto and grow;

wherein the microcarrier comprises a magnetoelastic sensor configured to vibrate with a specific resonant frequency when activated by a first magnetic field and generate a second magnetic field that can be remotely detected.
Example 10. The bioreactor of example 9, further comprises a drive coil configured to generate the first magnetic field to activate the magnetoelastic sensor and a detection coil configured to detect the second magnetic field generated by the magnetoelastic sensor.
Example 11. The bioreactor of example 10, wherein the first magnetic field comprises a DC magnetic field component and an AC magnetic field component, wherein the drive coil comprises a DC coil configured to generate the DC magnetic field component and an AC coil configured to generate the AC magnetic field component.
Example 12. The bioreactor of any one of examples 10-11, wherein the microcarrier is one of a plurality of microcarriers disposed inside the chamber, each microcarrier comprising a respective magnetoelastic sensor.
Example 13. The bioreactor of example 12, wherein the drive coil is configured to be movable relative to the chamber such that the magnetoelastic sensors in the plurality of microcarriers can be selectively activated by the first magnetic field generated by the drive coil.
Example 14. The bioreactor of any one of examples 12-13, wherein the detection coil is configured to be rotatable relative to the chamber such that the second magnetic field generated by the magnetoelastic sensors in the plurality of microcarriers can be selectively detected by the detection coil.
Example 15. The bioreactor of any one of examples 12-14, wherein the detection coil is one of a plurality of detection coils configured to simultaneously detect the second magnetic fields generated by the magnetoelastic sensors in the plurality of microcarriers.
Example 16. The bioreactor of any one of examples 12-15, the plurality of microcarriers are arranged in two or more planes inside the chamber.
Example 17. The bioreactor of any one of examples 10-16, wherein the magnetoelastic sensor is one of a plurality of magnetoelastic sensors having different resonant frequencies when they are exposed to the first magnet field, wherein the drive coil is configured to generate the first magnetic field having a frequency spectrum covering all resonant frequencies of the plurality of magnetoelastic sensors.
Example 18. A sensor for measuring cell growth on a microcarrier, the sensor comprising:

a magnetoelastic substrate;

a first coated layer encapsulating the magnetoelastic substrate, the first coated layer comprising a biocompatible material; and

a second coated layer encapsulating the first coated layer, the second coated layer being configured to promote cells to attach to and grow on the microcarrier.
Example 19. The sensor of example 18, wherein the biocompatible material in the first coated layer comprises parylene, and the second coated layer comprises polydopamine.
Example 20. The sensor of example 19, wherein the second coated layer comprises denatured collagen covalently linked to the polydopamine.
Example 21. The sensor of any one of examples 18-20, further comprises a third coated layer, wherein the third coated layer comprises a chemical configured to react to a target analyte surrounding the microcarrier such that reaction of the chemical to the target analyte changes elasticity of the third coated layer.
Example 22. The sensor of example 21, wherein the target analyte comprises hydrogen ion and the coated layer comprises polyacrylic acid.
Example 23. The sensor of any one of examples 21-22, wherein the target analyte comprises oxygen.
Example 24. The sensor of any one of examples 21-23, wherein the target analyte comprises glucose.
Example 25. The sensor of any one of examples 21-24, wherein the target analyte comprises a bacteria.
Example 26. A system for monitoring cell growth in a bioreactor, the system comprising:

a drive coil configured to generate a first magnetic field to activate a magnetoelastic sensor inside the bioreactor;

a detection coil configured to detect a second magnetic field generated by the magnetoelastic sensor after activated by the first magnetic field; and

a processing unit configured to analyze the second magnetic field to calculate one or more resonant response parameters of the magnetoelastic sensor.
Example 27. The system of example 26, wherein the drive coil comprises a pair of coil conductors disposed on opposite sides of the bioreactor.
Example 28. The system of example 27, wherein the pair of coil conductors have toroidal shape so as to generate the first magnetic field in a concentrated plane defined between the pair of coil conductors.
Example 29. The system of any one of examples 27-28, wherein the pair of coil conductors and the bioreactor are configured to be movable relative to each other in a direction that is perpendicular to an axis connecting the pair of coil conductors.
Example 30. The system of example 29, wherein the bioreactor is configured to be stationary and the pair of coil conductors are configured to be movable relative to the bioreactor.
Example 31. The system of example 29, wherein the pair of coil conductors are configured to be stationary and the bioreactor is configured to be movable relative to the pair of coil conductors.
Example 32. The system of any one of examples 26-31, wherein the detection coil and the bioreactor are configured to be rotatable relative to each other.
Example 33. The system of example 32, wherein the bioreactor is nonrotational and the detection coil is configured to be rotatable around to the bioreactor.
Example 34. The system of example 32, wherein the detection coil is nonrotational and the bioreactor is configured to be rotatable around the detection coil.
Example 35. The system of any one of examples 26-34, wherein the detection coil is one of a plurality of detection coils disposed on one side of the bioreactor.
Example 36. The system of example 35, wherein the plurality of detection coils are arranged in a two-dimensional grid, wherein the two-dimensional grid is located on a plane that is perpendicular to a direction of the second magnetic field.
Example 37. The system of example 36, wherein the plurality of coils are configured to shift position on the plane relative to the bioreactor, wherein a distance of positional shift along a shifting axis is smaller than a distance between two adjacent coils along the shifting axis.
Example 38. The system of any one of examples 35-37, further comprises a multiplexer connected to the plurality of detection coils so that the processing unit can sequentially analyze the second magnetic field detected by each of the plurality of detection coils.
Example 39. The system of any one of examples 35-38, wherein the drive coil is configured to generate the first magnetic field having a frequency spectrum defined by a plurality of resonant frequencies respectively corresponding to a plurality of magnetoelastic sensors disposed inside the bioreactor.
Example 40. The system of example 39, wherein the processing unit is configured to determine an origin of the second magnetic field detected by the plurality of detection coils by finding a maximum signal strength of the detected second magnetic field among the plurality of detection coils.
Example 41. The system of any one of examples 26-40, further comprises an amplifier configured to amplify a signal corresponding to the detected second magnetic field.
Example 42. The system of example 41, wherein the amplifier comprises a bandpass filter having a center frequency corresponding to a resonant frequency of the magnetoelastic sensor and a predefined bandwidth configured to suppress signals from other magnetoelastic sensors.
Example 43. The system of any of examples 26-42, further comprises a drive circuit configured to generate an AC drive current and a DC biasing current.
Example 44. The system of example 43, wherein the drive circuit is configured to alternate between an ON phase and an OFF phase, wherein in the ON phase, the drive circuit is configured to generate the AC drive current for a plurality of drive cycles, and wherein in the OFF phase, the drive circuit generates no AC drive current.
Example 45. The system of example 44, wherein a duration of the OFF phase is determined based on a number of detection coils in the system.
Example 46. The system of any one of examples 26-45, wherein the one or more resonant response parameters comprise a resonant frequency of the magnetoelastic sensor.
Example 47. The system of example 46, wherein the processing unit is configured to detect a shift of the resonant frequency measured at two different points in time.
Example 48. The system of any one of examples 46-47, wherein the one or more resonant response parameters comprise a phase angle of impedance at the resonant frequency.
Example 49. The system of example 48, wherein the processing unit is configured to detect a shift of the phase angle of impedance measured at two different points in time.
Example 50. The system of any one of examples 46-49, wherein the one or more resonant response parameters comprise a magnitude of impedance at the resonant frequency.
Example 51. The system of example 50, wherein the processing unit is configured to detect a shift of the magnitude of impedance measured at two different points in time.
Example 52. The system of any one of examples 46-51, wherein the one or more resonant response parameters comprise a resonance quality factor.
Example 53. The system of example 52, wherein the processing unit is configured to detect a shift of the resonance quality factor measured at two different points in time.
Example 54. The system of any one of examples 26-53, further comprises a computer readable media storing at least one calibration curve associated with the magnetoelastic sensor.
Example 55. The system of example 54, wherein the processing unit is configured to compare the one or more resonant response parameters with the at least one calibration curve to generate a count of cells attached to the magnetoelastic sensor.
Example 56. The system of any one of examples 54-55, wherein the processing unit is configured to compare the one or more resonant response parameters with the at least one calibration curve to generate a chemical concentration surrounding the magnetoelastic sensor.
Example 57. The system of example 56, wherein the processing unit is configured to compare the one or more resonant response parameters with the at least one calibration curve to generate a pH surrounding the magnetoelastic sensor.
Example 58. The system of any one of examples 54-56, wherein the processing unit is configured to compare the one or more resonant response parameters with the at least one calibration curve to detect at least one biomarker surrounding the magnetoelastic sensor.
Example 59. The system of example 58, wherein the at least one biomarker comprises glucose.
Example 60. The system of any one of examples 26-59, wherein the processing unit is configured to track a variation of the one or more resonant response parameters over a period of time.
Example 61. A method of fabricating a cell sensing apparatus, the method comprising:

embedding a magnetoelastic sensor inside a microcarrier configured for cells to attached thereto;

wherein the magnetoelastic sensor is configured to vibrate with a specific resonant response when exposed to a first magnetic field and generate a second magnetic field.
Example 62. The method of example 61, further comprises fabricating the magnetoelastic sensor using a magnetoelastic substrate.
Example 63. The method of example 62, further comprises preparing the magnetoelastic substrate by electrodepositing a layer of magnetoelastic material over a polymer substrate.
Example 64. The method of example 63, wherein preparing the magnetoelastic substrate comprises depositing a photoresist material to the layer of magnetoelastic material and masking the layer of magnetoelastic material with one or more masks having a predefined size.
Example 65. The method of example 64, wherein preparing the magnetoelastic substrate comprises applying an ultraviolet light to the magnetoelastic substrate.
Example 66. The method of example 65, wherein preparing the magnetoelastic substrate comprises using plasma etching to remove the magnetoelastic material unmasked by the one or more masks.
Example 67. The method of example 66, wherein preparing the magnetoelastic substrate comprises applying an organic solvent to remove the photoresist material and the polymer substrate.
Example 68. The method of any one of examples 62-67, wherein fabricating the magnetoelastic sensor comprises cutting the magnetoelastic substrate into a segment having a predefined dimension.
Example 69. The method of any one of examples 62-68, wherein fabricating the magnetoelastic sensor comprises cleaning the magnetoelastic substrate with a cleaning solution, air-drying the magnetoelastic substrate, and annealing the magnetoelastic substrate at a predefined temperature for a predefined duration.
Example 70. The method of any one of examples 62-69, wherein fabricating the magnetoelastic sensor comprises coating the magnetoelastic substrate with a first layer, wherein the first layer comprises a biocompatible material.
Example 71. The method of claim 70, wherein fabricating the magnetoelastic sensor comprises altering surface roughness of the first layer by means of reactive ion etching.
Example 72. The method of any one of examples 70-71, wherein fabricating the magnetoelastic sensor comprises sterilizing the first layer.
Example 73. The method of any one of examples 70-72, wherein fabricating the magnetoelastic sensor comprises encapsulating the first layer with a second layer, wherein the second layer comprises polydopamine.
Example 74. The method of example 73, wherein fabricating the magnetoelastic sensor comprises coating the second layer with a third layer, wherein the third layer comprises a chemical configured to react to an analyte surrounding the microcarrier such that reaction of the chemical to the analyte changes elasticity of the microcarrier, thereby changing the magnetoelastic sensor's resonant response.
Example 75. The method of any one of examples 73-74, wherein fabricating the magnetoelastic sensor comprises encapsulating the magnetoelastic sensor within a layer of denatured collagen so that the denatured collagen and the polydopamine are covalently linked to each other.
Example 76. The method of any one of example 61-75, wherein the magnetoelastic sensor is a first magnetoelastic sensor, wherein the method further comprises embedding a second magnetoelastic sensor inside the microcarrier, wherein the first and second magnetoelastic sensors vibrate at different resonant frequencies when they are exposed to the first magnet field.
Example 77. The method of any of examples 61-76, further comprises placing the microcarrier inside a bioreactor configured to grow cells therein.
Example 78. The method of example 77, further the microcarrier is one of a plurality of microcarriers, wherein each microcarrier comprises at least one magnetoelastic sensor, and the method further comprises placing the plurality of microcarriers inside the bioreactor.
Example 79. The method of example 77, wherein the plurality of microcarriers are arranged in three-dimensional space of the bioreactor.
Example 80. The method of example 79, further comprises placing the plurality of microcarriers in two or more parallel layers inside the bioreactor.
Example 81. The method of any one of examples 77-80, further comprises placing a drive coil outside the bioreactor to generate the first magnetic field and placing a detection coil outside the bioreactor to detect the second magnetic field, wherein a drive circuit is configured to provide an excitation current to the drive coil, and a detection circuit is configured to process a signal detected by the detection coil.
Example 82. The method of example 81, wherein the detection coil and the drive coil are the same and the drive circuit is isolated from the detection circuit.
Example 83. The method of example 81, wherein the detection coil is one of a plurality of detection coils arranged in a planar surface.
Example 84. The method of example 83, further comprises connecting the plurality of detection coils to a multiplexer that is operationally connected to a signal processing unit configured to analyze the second magnetic field detected by each of the plurality of detection coils.
Example 85. A method for monitoring cells in a bioreactor, the method comprising:

placing a microcarrier inside the bioreactor, wherein the microcarrier is configured for cells to attach thereto and embeds a magnetoelastic sensor;

exposing the microcarrier to a first magnetic field so that the magnetoelastic sensor can vibrate with a resonant frequency and generate a second magnetic field; and

detecting the second magnetic field and measuring a resonant response of the magnetoelastic sensor.
Example 86. The method of example 85, further comprises calculating a mass of cells attached to the magnetoelastic sensor based on the measured resonant response of the magnetoelastic sensor.
Example 87. The method of any one of examples 85-86, further comprises calculating a chemical concentration surrounding the magnetoelastic sensor based on the measured resonant response of the magnetoelastic sensor.
Example 88. The method of any one of examples 85-87, further comprises tracking cell growth in the bioreactor over a period of time based on continuous measurement of the resonant response of the magnetoelastic sensor.
Example 89. The method of any one of examples 85-88, wherein the magnetoelastic sensor is a first magnetoelastic sensor, and the method further comprises embedding a second magnetoelastic sensor inside the bioreactor, wherein the first and second magnetoelastic sensors vibrate with different resonant frequencies when exposed to the first magnetic field.
Example 90. The method of example 89, further comprises generating the first magnetic field having at least two frequencies that respectively correspond to the resonant frequencies of the first and second magnetoelastic sensors so that the first and second magnetoelastic sensors can be simultaneously activated at their respective resonant frequencies by the first magnetic field.
Example 91. The method of example 90, further comprises simultaneously measuring resonant responses of the first and second magnetoelastic sensors from a mixed signal containing the second magnetic field generated by each of the first and second magnetoelastic sensors.
Example 92. The method of any one of examples 85-91, wherein the microcarrier is a first microcarrier, and the method further comprises placing a second microcarrier inside the bioreactor, wherein the second microcarrier comprises at least another magnetoelastic sensor.
Example 93. The method of example 92, further comprises selectively exposing the first and second microcarriers to the first magnetic field so as to selectively activate respective magnetoelastic sensors embedded in the first and second microcarriers.
Example 94. The method of example 93, wherein sequentially exposing the first and second microcarriers to the first magnetic field comprises actuating relative movement between a drive coil and the bioreactor in the specified direction so that the first magnetic field generated by the drive coil passes through the first and second microcarriers sequentially.
Example 95. The method of any one of examples 93-94, further comprises actuating relative rotation between a detection coil and the bioreactor so that the second magnetic field generated by respective magnetoelastic sensors in the first and second microcarriers can be sequentially detected by the detection coil.
Example 96. The method of any one of examples 92-95, further comprises detecting the second magnetic field generated by respective magnetoelastic sensors in the first and second microcarriers using a plurality of detection coils located on one side of the bioreactor, wherein the plurality of detection coils are arranged in a planar surface.
Example 97. The method of example 96, wherein the method further comprises shifting positions of the plurality of detection coils on the planar surface relative to the bioreactor.
Example 98. A method comprising:

placing a microcarrier inside a bioreactor, wherein the microcarrier is configured for cells to attach thereto and embeds a magnetoelastic sensor;

exposing the microcarrier to a first activation magnetic field so that the magnetoelastic sensor can vibrate with a first amplitude, wherein vibration of the microcarrier at the first amplitude is configured to modulate growth of the cells attached to the microcarrier.
Example 99. The method of example 98, further comprises exposing the microcarrier to a second activation magnetic field so that the magnetoelastic sensor can vibrate with a second amplitude, wherein the first amplitude is at least 10 times the second amplitude.
Example 100. The method of any one of examples 98-99, further comprises detecting a return magnetic field generated by the magnetoelastic sensor in response to the exposure to the second activation magnetic field and measuring a resonant response of the magnetoelastic sensor.

### Example Alternatives

The technologies from any example can be combined with the technologies described in any one or more of the other examples. In view of the many possible examples to which the principles of the disclosed technology can be applied, it should be recognized that the illustrated embodiments are examples of the disclosed technology and should not be taken as a limitation on the scope of the disclosed technology. Rather, the scope of the claimed subject matter is defined by the following claims and their equivalents.

1. An apparatus, comprising:
a microcarrier configured to receive and support attachment and growth of cells; and
a magnetoelastic sensor enclosed by the microcarrier and configured to vibrate in response to an activation magnetic field,
wherein the vibration produces a return magnetic field having detectable field characteristics associated with the attachment or growth of the cells.

2. The apparatus of claim 1, wherein the microcarrier is porous.

3. The apparatus of claim 1, wherein the magnetoelastic sensor is one of a plurality of magnetoelastic sensors, wherein the plurality of magnetoelastic sensors have different lengths so that they vibrate with different resonant responses when they are exposed to the activation magnet field.

4. The apparatus of claim 1, wherein the magnetoelastic sensor comprises a magnetoelastic substrate, wherein the magnetoelastic substrate has a rod or a rectangular strip shape.

5. The apparatus of claim 4, wherein the magnetoelastic substrate has an aspect ratio between 2 and 4, wherein the aspect ratio is calculated as a ratio of an axial length of the magnetoelastic substrate to a maximum width in a cross-section that is perpendicular to the axial length of the magnetoelastic substrate.

6. The apparatus of claim 4, wherein the magnetoelastic sensor comprises a first coated layer encapsulating the magnetoelastic substrate, wherein the first coated layer comprises a biocompatible material.

7. The apparatus of claim 6, wherein the magnetoelastic sensor comprises a second coated layer encapsulating the first coated layer, wherein the second coated layer is configured to promote attachment of cells to the microcarrier without destructing the cells.

8. The apparatus of claim 7, wherein the magnetoelastic sensor comprises a third coated layer, wherein the third coated layer comprises a chemical configured to react to an analyte surrounding the microcarrier such that reaction of the chemical to the analyte causes shrink or swell of the third coated layer, thereby changing the magnetoelastic sensor's resonant response.

9. An apparatus, comprising:
a bioreactor chamber configured to grow cells; and
a microcarrier disposed inside the bioreactor chamber and configured to support attachment and growth of the cells,
wherein the microcarrier comprises a magnetoelastic sensor configured to vibrate with a specific resonant frequency when activated by a first magnetic field and to produce a remotely detectable second magnetic field from the vibration.

10. The apparatus of claim 9, further comprises a drive coil configured to generate the first magnetic field to activate the magnetoelastic sensor and a detection coil configured to detect the second magnetic field generated by the magnetoelastic sensor.

11. The apparatus of claim 10, wherein the first magnetic field comprises a DC magnetic field component and an AC magnetic field component, wherein the drive coil comprises a DC coil configured to generate the DC magnetic field component and an AC coil configured to generate the AC magnetic field component.

12. The apparatus of claim 10, wherein the microcarrier is one of a plurality of microcarriers disposed inside the bioreactor chamber, each microcarrier comprising a respective magnetoelastic sensor.

13. The apparatus of claim 12, wherein the drive coil is configured to be movable relative to the bioreactor chamber such that the magnetoelastic sensors in the plurality of microcarriers can be selectively activated by the first magnetic field generated by the drive coil.

14. The apparatus of claim 12, wherein the detection coil is configured to be rotatable relative to the bioreactor chamber such that the second magnetic field generated by the magnetoelastic sensors in the plurality of microcarriers can be selectively detected by the detection coil.

15. The apparatus of claim 12, wherein the detection coil is one of a plurality of detection coils configured to simultaneously detect the second magnetic fields generated by the magnetoelastic sensors in the plurality of microcarriers.

16. The apparatus of claim 12, wherein the plurality of microcarriers are arranged in two or more planes inside the bioreactor chamber.

17. The apparatus of claim 10, wherein the magnetoelastic sensor is one of a plurality of magnetoelastic sensors having different resonant frequencies when they are exposed to the first magnet field, wherein the drive coil is configured to generate the first magnetic field having a frequency spectrum containing all resonant frequencies of the plurality of magnetoelastic sensors.

18. An apparatus, comprising:
a microcarrier including a magnetoelastic substrate,
a first coated layer encapsulating the magnetoelastic substrate and comprising a biocompatible material, and
a second coated layer encapsulating the first coated layer,
wherein the second coated layer is configured to promote cells to attach to and grow on the microcarrier.

19. The apparatus of claim 18, wherein the biocompatible material in the first coated layer comprises parylene, and the second coated layer comprises polydopamine and denatured collagen covalently linked to the polydopamine.

20. The apparatus of claim 18, further comprises a third coated layer, wherein the third coated layer comprises a chemical configured to react to a target analyte surrounding the microcarrier such that reaction of the chemical to the target analyte changes elasticity of the third coated layer.
